(19) United States
(12) Patent Application Publication
(10) Pub. No.: US 2014/0011185 A1
(43) Pub. Date: Jan. 9, 2014

(54) METHODS AND SYSTEMS FOR CONTROLLING GROWTH RATES OF AUTOTROPHIC MICROBIAL CULTURES

(71) Applicant: Peter Stroot, Parker, CO (US)

(72) Inventor: Peter Stroot, Parker, CO (US)

(21) Appl. No.: 13/929,547

(22) Filed: Jun. 27, 2013

Related U.S. Application Data
(60) Provisional application No. 61/666,557, filed on Jun. 29, 2012.

(51) Int. Cl. C12Q 3/00

(52) U.S. Cl. CPC C12Q 3/00
USPC 435/3; 435/286.1

(57) ABSTRACT

The present application describes a method of regulating CO₂ concentrations of bioreactor systems to regulate the specific growth rate of various autotrophic microbes for cultivation or bioprocessing of liquids and gases.

### CROSS-REFERENCE TO RELATED APPLICATIONS

This application claims the priority benefit under 35 U.S.C. §119(e) of U.S. Provisional Application No. 61/666,557, filed Jun. 29, 2012, the entirety of which is hereby incorporated by reference herein.

### BACKGROUND

1. Field

This application relates generally to human health, animal health, biomining, soil bioremediation, biochemical production and/or the like, and more specifically, to methods, devices and systems of at least partially improving or optimizing CO₂ levels in mixtures and/or other environments to increase the specific growth rate of aerobic and anaerobic autotrophic microbes.

2. Description of the Related Art

The growth of nitrifying bacteria, Anammox bacteria, and methanogens can be sensitive to the soluble carbon dioxide (sCO₂) concentration in bioreactor systems used for treating wastewater and sludge. Beyond these mixed culture systems, it does not appear that autotrophic microorganisms have been fully exploited for biomedical, agricultural, industrial, environmental and/or other applications or uses due to, among other things, the difficulty in cultivating pure cultures. The disclosure provides guidance on methods for generating pure cultures of autotrophic microbes for biomedical and biotechnological applications. In some cases, these types of systems (e.g., bioreactors) may be of interest for the generation of valuable endproducts. Guidance is also provided on the evaluation and improvement of autotrophic growth conditions in bioreactor systems treating wastewater, sludge and/or other waste products.

### SUMMARY

According to some embodiments, a method for controlling the growth of autotrophic cells in a bioreactor includes determining a concentration of soluble carbon dioxide within a liquid mixture of the bioreactor, wherein the bioreactor comprises a volume of the liquid mixture below a headspace of the bioreactor, and wherein said liquid mixture comprises autotrophic cells and substrate, said substrate being configured to promote the growth of the autotrophic cells when the bioreactor is in operation. The method additionally comprises calculating a target range of a concentration of soluble carbon dioxide within the liquid mixture based on, at least in part, on empirical or experimental data, wherein the target range provides for controlled (e.g., increased or enhanced, suppressed or inhibited, etc.) growth of the autotrophic cells when the bioreactor is in use, and comparing the target range of the concentration of soluble carbon dioxide within the liquid mixture to the concentration of soluble carbon dioxide in the liquid mixture. The method comprises adjusting the concentration of soluble carbon dioxide within the liquid mixture by at least one of: (i) modifying a partial pressure of carbon dioxide gas within the headspace, and (ii) modifying the concentration of soluble carbon dioxide within the liquid mixture by delivering a volume of a supplement stream to the liquid mixture. In some embodiments, a concentration of soluble carbon dioxide in the supplement stream is different than the concentration of soluble carbon dioxide of the liquid mixture.

According to some embodiments, the headspace is not in fluid communication with an ambient environment, such that an interior of the bioreactor comprises a closed system. In one embodiment, the bioreactor comprises a cover or some other enclosure device to isolate the liquid mixture and the headspace from the outside (e.g., ambient) environment. In some embodiments, the headspace is in fluid communication with an ambient environment, such that the bioreactor comprises an open system. In one embodiment, the bioreactor comprises a partially open cover or no cover at all.

According to some embodiments, the method additionally includes measuring a temperature of the liquid mixture, wherein the measured temperature of the liquid mixture is used, at least in part, to calculate the target range of a concentration of soluble carbon dioxide. In some embodiments, the method further comprises modifying a temperature of the liquid mixture (e.g., via one or more heating and/or cooling devices or systems) to achieve a target temperature for the liquid mixture, wherein the target temperature assists in enhancing the growth of the autotrophic cells. According to some embodiments, the method further comprises measuring a pH of the liquid mixture, wherein the measured pH is used, at least in part, to calculate the target range of a concentration of soluble carbon dioxide. In some embodiments, the method further comprises modifying a pH of the liquid mixture (e.g., using an injection or fluid delivery system) to achieve a target pH for the liquid mixture, wherein the target pH assists in enhancing the growth of the autotrophic cells.

According to some embodiments, modifying the partial pressure of carbon dioxide gas within the headspace comprises introducing a volume of gas within the headspace of the bioreactor, the volume of gas comprising a concentration of carbon dioxide that is different (e.g., higher or lower) than a concentration of carbon dioxide gas within said headspace. In some embodiments, the gas introduced within the headspace comprises an inert gas having little or no carbon dioxide (e.g., N2, ambient air, etc.).

According to some embodiments, determining the concentration of soluble carbon dioxide within the liquid mixture comprises using a probe, sensor or other measurement device or system. Such a probe or other device or system can be incorporated in the bioreactor system or can be separate and distinct from the bioreactor. In some embodiments, a separate and distinct probe or sensor is in data communication with a control system for the bioreactor. In some embodiments, the probe is inserted or positioned within the liquid mixture to directly determine the concentration of soluble carbon dioxide. In some embodiments, the probe comprises a carbon dioxide probe or sensor. In some embodiments, determining the concentration of soluble carbon dioxide within the liquid mixture comprises calculating the concentration of soluble carbon dioxide using an empirical relationship between soluble carbon dioxide and at least one property of the liquid mixture. In some embodiments, the at least one property of the liquid mixture that is used to calculate the concentration of soluble carbon dioxide comprises pH, total alkalinity, temperature and/or any other property, input or consideration. In some embodiments, measuring the concentration of soluble carbon dioxide with the liquid mixture comprises calculating the concentration of soluble carbon dioxide based on, at least in part, a measured partial pressure of carbon dioxide gas within the headspace, a temperature of the liquid mixture and Henry's constant for carbon dioxide.

According to some embodiments, a supplemental stream of autotrophic cells is configured to be selectively delivered to the bioreactor, wherein the supplemental stream is contained within a supplemental container. In one embodiment, the supplemental stream comprises substrate (e.g., carbon source for cell growth, minerals, nutrients, etc.), wherein the substrate is configured to promote the growth of the autotrophic cells. In some embodiments, the method additionally comprises measuring a concentration of soluble carbon dioxide within the supplemental stream contained within the supplemental container, calculating a target range of a concentration of soluble carbon dioxide within the supplemental stream based on, at least in part, on empirical or experimental data, comparing the target range of the concentration of soluble carbon dioxide within the supplemental liquid to the measured concentration of soluble carbon dioxide in the supplemental liquid, and adjusting the concentration of soluble carbon dioxide within the supplemental liquid by modifying the concentration of carbon dioxide gas within the headspace.

According to some embodiments, the autotrophic cells comprise nitrifying bacteria. In some embodiments, the method further includes adjusting a concentration of ammonium and/or a concentration of nitrite within the liquid mixture to ensure that a growth of the nitrifying bacteria is not nitrogen limited.

According to some embodiments, the autotrophic cells comprise phototrophic microbes. In some embodiments, the autotrophic cells comprise sulfide oxidizing bacteria. In some embodiments, the autotrophic cells comprise precious metal precipitating bacteria. In some embodiments, the method additionally comprises increasing a concentration of hydrogen in the headspace of the bioreactor by injecting hydrogen-rich gas into the headspace in order to promote the growth of the precious metal precipitating bacteria. In one embodiment, the method further includes increasing a dissolved oxygen concentration in the liquid mixture when the autotrophic cells comprise aerobic microbes. In some embodiments, increasing the dissolved oxygen concentration in the liquid mixture comprises injecting an oxygen-laden gas into at least one of said liquid mixture and said headspace.

According to some embodiments, the autotrophic cells comprise Anammox bacteria. In some embodiments, the method further comprises adjusting a concentration of ammonium and/or a concentration of nitrite within the liquid mixture to ensure that a growth of the Anammox bacteria is not nitrogen limited.

According to some embodiments, the method further comprises adjusting an oxidation reduction potential within the liquid mixture by adding a volume of a solution to the liquid mixture, wherein the volume of the solution comprises a concentration of reducing agent that is different than a concentration of reducing agent within the liquid mixture.

According to some embodiments, the autotrophic cells comprise CO2-reducing methanogens. In some embodiments, the autotrophic cells comprise acetogens. In some embodiments, the method additionally includes maintaining a concentration of acetate within the liquid mixture below a threshold level of acetate to ensure proper growth of the acetogens. In some embodiments, the autotrophic cells comprise aceticlastic methanogens. In some embodiments, the method additionally includes maintaining a concentration of acetate within the liquid mixture above a threshold level of acetate to ensure proper growth of the aceticlastic methanogens.

According to some embodiments, the autotrophic cells comprise syntrophic bacteria and CO2-reducing methanogens. In some embodiments, the method further comprises maintaining a concentration of propionate within the liquid mixture above a threshold level of propionate to ensure proper growth of the autotrophic cells. In some embodiments, a mixing intensity within the bioreactor is maintained below a threshold mixing intensity level in order to promote co-localization of the syntrophic bacteria vis-à-vis the CO2-reducing methanogens.

According to some embodiments, the autotrophic cells comprise Syngas-fermenting bacteria. In some embodiments, the method additionally includes adjusting a partial pressure of carbon monoxide within the headspace. In some embodiments, the autotrophic cells comprise dehalogenating bacteria. In one embodiment, the method further comprises maintaining a concentration of halogenated organics within the liquid mixture above a threshold level to ensure proper growth of the dehalogenating bacteria.

According to some embodiments, the autotrophic cells comprise short chain fatty acid methylating microbes. In some embodiments, the method additionally includes maintaining a concentration of substrate short chain fatty acid within the liquid mixture above a threshold level to ensure proper growth of short chain fatty acid methylating microbes. In some embodiments, the autotrophic cells comprise alkane methylating microbes. In one embodiment, the method further comprises maintaining a concentration of substrate alkane within the liquid mixture above a threshold level to ensure proper growth of alkane methylating microbes.

According to some embodiments, the autotrophic cells comprise alcohol methylating bacteria. In some embodiments, the method further comprises maintaining a concentration of substrate alcohol within the liquid mixture above a threshold level to ensure proper growth of alcohol methylating bacteria.

According to some embodiments, it is desirable to intentionally inhibit or suppress growth of autotrophic cells in a bioreactor. For example, it may be desirable in a wastewater treatment train to provide poor growth conditions for autotrophs (e.g., elevated sCO2 concentration) to enhance or promote the operational performance of such a bioreactor (e.g., to selectively promote the growth of other types of cells, e.g., non-autotrophic cells).

According to some embodiments, the bioreactor comprises a stand-alone system for producing autotrophic cells. In some embodiments, the bioreactor is incorporated into an engineered biological system (e.g., wastewater treatment system, sludge/biosolids treatment system, other liquid, gas or solid treatment systems, biochemical production systems and/or the like). In some embodiments, the bioreactor is in fluid communication with a treatment chamber of the wastewater treatment system so that autotrophic cells grown within the bioreactor can be selectively delivered into the treatment chamber.

According to some embodiments, a bioreactor for controlling the growth of autotrophic cells comprises at least one chamber (e.g., tank, container, etc.) for retaining a liquid mixture, an inlet and an outlet in fluid communication with the at least one chamber, wherein the inlet in configured to permit a liquid mixture to enter the bioreactor, and wherein the outlet is configured to permit a liquid mixture to exit the bioreactor and a headspace located above the chamber and the liquid mixture. In some embodiments, the bioreactor comprises at least one probe or sensor configured to determine a concentration of soluble carbon dioxide within a liquid mixture of the bioreactor. The concentration of soluble carbon dioxide can be determined directly (e.g., by using a carbon dioxide sensor or probe) and/or indirectly (e.g., by using an empirical formula that takes into consideration other measured or detected properties of the liquid mixture, headspace and/or the like).

According to some embodiments, the bioreactor further comprises a gas regulation system configured to permit a gas to be selectively moved within the headspace of the bioreactor, wherein the gas regulation system is configured to: (i) alter a concentration of carbon dioxide of the gas moved within the headspace and/or (ii) alter a flowrate of the gas moved within the headspace. The bioreactor can additionally include a control system for regulating a concentration of soluble carbon dioxide within the liquid mixture, wherein the control system is configured to determine a target range of a concentration of soluble carbon dioxide within the liquid mixture based on, at least in part, on empirical or experimental data, wherein the target range provides for controlled (e.g., enhanced or increased growth, suppressed or inhibited growth, etc.) growth of the autotrophic cells when the bioreactor is in use. In some embodiments, the control system is configured to compare the target range of the concentration of soluble carbon dioxide within the liquid mixture to the concentration of soluble carbon dioxide in the liquid mixture. In some embodiments, the control system is configured to adjust the concentration of soluble carbon dioxide within the liquid mixture by at least one of: (i) modifying a partial pressure of carbon dioxide gas within the headspace, and (ii) modifying the concentration of soluble carbon dioxide within the liquid mixture by delivering a volume of a supplement stream to the liquid mixture;

According to some embodiments, the headspace is not in fluid communication with an ambient environment, such that an interior of the bioreactor comprises a closed system. In one embodiment, the bioreactor comprises an upper cover, lid or other enclosure. In some embodiments, the bioreactor comprises an upper enclosure or cover above the liquid mixture. In some embodiments, the headspace is in fluid communication with an ambient environment, such that the bioreactor comprises an open system (e.g., the bioreactor does not include a cover or other enclosure).

According to some embodiments, the bioreactor further comprises at least one additional probe or sensor (e.g., to measure at least one of a temperature, pH, alkalinity, soluble carbon dioxide, etc. of the liquid mixture). In some embodiments, the bioreactor is incorporated into a wastewater treatment system. In some embodiments, the bioreactor comprises an activated sludge treatment tank and/or a digester (e.g., anaerobic digester or system) included in a treatment scheme. In some embodiments, the bioreactor is in fluid communication with an activated sludge treatment tank and/or a digester (e.g., anaerobic digester) included in a treatment scheme.

According to some embodiments, the various systems and methods disclosed herein can be used for the production of microbial biomass for a variety of applications, including, but not limited to: bioaugmenting wastewater and sludge treatment systems; improving phototrophic microbial biomass production rate; reducing hydrogen gas and propionic acid in the large intestine of humans and animals by novel probiotics; reducing startup and improving the efficiency of biomining reactor systems; improving dehalogenation rates of pollutants in soil; generating propionic acid and butyric acid in ruminants for the reduction of methane generate by novel probiotics, producing biochemicals through novel methylation reactions and/or the like. Methods and systems are described for the cultivation of pure cultures of autotrophic microbes. Additional guidance is provided for cultivating novel autotrophic microbes for probiotics and biofuels. Finally, guidance is provided for evaluating and improving the growth conditions for autotrophic microbes in wastewater and sludge treatment systems that may be targeted for bioaugmentation.

### BRIEF DESCRIPTION OF THE DRAWINGS

These and other features, aspects and advantages of the present inventions are described with reference to drawings of certain preferred embodiments, which are intended to illustrate, but not to limit, the present inventions. It is to be understood that the attached drawings are provided for the purpose of illustrating concepts of the present inventions and may not be to scale.

FIG. 1 is a diagram showing one embodiment of a method for controlling pCO₂ in the headspace of a bioreactor for cultivation of pure autotrophic culture(s).

FIG. 2 illustrates a diagram showing one embodiment of a method for controlling pCO₂ in the headspace of a bioreactor for cultivation of pure autotrophic culture.

FIG. 3 is a chart that compares the pH as a function of sCO₂ concentration for total alkalinities of 120 and 250 mg/L as CaCO₃.

FIG. 4 is a chart that describes the sCO₂ concentration as a function of pH for total alkalinity of 120 mg/L as CaCO₃.

FIG. 5 illustrates a diagram showing one embodiment of a method for controlling sCO₂ of a bioreactor by pH control for cultivation of pure autotrophic culture with a constant total alkalinity.

FIG. 6 is a diagram showing one embodiment of a modified reactor configuration for the cultivation of nitrifying bacteria that grow rapidly with optimal sCO₂ by control of the headspace pCO₂ concentration.

FIG. 7 is a diagram showing one embodiment of a modified reactor configuration for the cultivation of phototrophic, autotrophic microbes, such as, for example, Cyanobacteria, that grow rapidly with optimal or otherwise enhanced sCO₂ by control of the headspace pCO₂ concentration.

FIG. 8 is a diagram showing one embodiment of a modified reactor configuration for the cultivation of sulfide oxidizing microbes that grow rapidly with optimal sCO₂ by control of the headspace pCO₂ concentration.

FIG. 9 is a diagram showing one embodiment of a modified reactor configuration for the cultivation of metals precipitating microbes that grow rapidly with optimal sCO₂ by control of the headspace pCO₂ concentration.

FIG. 10 is a diagram showing one embodiment of a modified reactor configuration for the cultivation of Anammox bacteria that grow rapidly with optimal or enhanced sCO₂ by control of the headspace pCO₂ concentration.

FIG. 11 is a diagram showing one embodiment of a modified reactor configuration for the cultivation of CO₂-reducing methanogens with optimal or enhanced sCO₂ by control of the headspace pCO₂ concentration.

FIG. 12 is a diagram showing one embodiment of a modified reactor configuration for the cultivation of acetogens with optimal or enhanced sCO₂ by control of the headspace pCO₂ concentration.

FIG. 13 is a diagram showing one embodiment of a modified reactor configuration for the cultivation of aceticlastic methanogens with optimal or enhanced sCO₂ by control of the headspace pCO₂ concentration.

FIG. 14 is a diagram showing one embodiment of a modified UASB reactor configuration for the cultivation of a co-culture of syntrophic bacteria and CO₂-reducing methanogens with optimal or enhanced sCO₂ by control of the headspace pCO₂ concentration.

FIG. 15 is a diagram showing one embodiment of a modified reactor configuration for the cultivation of autotrophic microbes that ferment Syngas that grow rapidly with optimal or enhanced sCO₂ by control of the headspace pCO₂ concentration.

FIG. 16 is a diagram showing one embodiment of a modified reactor configuration for the cultivation of autotrophic, dehalogenating bacteria that grow rapidly with optimal or enhanced sCO₂ by control of the headspace pCO₂ concentration.

FIG. 17 is a diagram showing one embodiment of a modified reactor configuration for the cultivation of autotrophic microbes that generate SCFA₍ₙ₊₁₎ by methylation that grow rapidly with optimal or enhanced sCO₂ by control of the headspace pCO₂ concentration.

FIG. 18 is a diagram showing one embodiment of a modified reactor configuration for the cultivation of autotrophic microbes that generate Alkane₍ₙ₊₁₎ by methylation that grow rapidly with optimal or enhanced sCO₂ by control of the headspace pCO₂ concentration.

FIG. 19 is a diagram showing one embodiment of a modified reactor configuration for the cultivation of autotrophic microbes that generate Alcohol₍ₙ₊₁₎ by methylation that grow rapidly with optimal or enhanced sCO₂ by control of the headspace pCO₂ concentration.

FIG. 20 is a diagram showing one embodiment of a modified reactor configuration for the cultivation of methanogens that grow rapidly with optimal or enhanced sCO₂ by control of the headspace pCO₂ concentration that are used to bioaugment an anaerobic digester and increase the methane content of the biogas in the headspace of the anaerobic digester.

FIG. 21 is a chart that describes the specific growth rate of four different nitrifying bacteria for a range of sCO₂ concentrations and a total alkalinity of 120 mg/L as CaCO₃.

FIG. 22 is a chart that describes the specific growth rate of four different nitrifying bacteria for a range of sCO₂ concentrations and a total alkalinity of 250 mg/L as CaCO₃.

FIG. 23 is a diagram showing one embodiment of a modified wastewater treatment train for biological nitrogen removal by the cultivation of ammonium oxidizing bacteria and Anammox bacteria that grow rapidly with optimal sCO₂ by control of the headspace pCO₂ concentration for a measured total alkalinity and pH.

FIG. 24 is a diagram showing one embodiment of a modified wastewater treatment train for biological nutrient removal by cultivation of ammonium oxidizing bacteria and Anammox bacteria that grow rapidly with optimal sCO₂ by control of the headspace pCO₂ concentration.

FIG. 25 is a diagram showing one embodiment of a modified wastewater treatment train for biomethane generation and biological nutrient removal by cultivation of autotrophic microbes that grow rapidly with optimal sCO₂ by control of the headspace pCO₂ concentration.

### DETAILED DESCRIPTION

In the following detailed description, reference is made to the accompanying drawings, which form a part hereof, and within which are shown by way of illustration specific embodiments by which the inventions disclosed herein may be practiced. It is to be understood that other embodiments may be utilized and structural changes may be made without departing from the scope of the inventions disclosed herein.

All numerical designations, such as pH, temperature, time, concentration, and molecular weight, including ranges, are approximations which are varied up or down by increments of 1.0 or 0.1, as appropriate. It is to be understood, even if it is not always explicitly stated that all numerical designations are preceded by the term “about” or “approximately”. It is also to be understood, even if it is not always explicitly stated, that the reagents described herein are merely included as examples. As such the use of specific reagents and/or materials or components included in a particular system can be substituted for any other reagents, materials and/or components even if not explicitly disclosed herein.

The systems, devices and method disclosed herein can be used in a variety of different systems across various spectra of applications and/or industries, including without limitation, wastewater treatment, industrial application, laboratories, pharmaceutical or other scientific fields and/or the like.

According to some embodiments, a method for controlling the growth of autotrophic cells in a bioreactor includes determining a concentration of soluble carbon dioxide within a liquid mixture of the bioreactor, wherein the bioreactor comprises a volume of the liquid mixture below a headspace of the bioreactor, and wherein said liquid mixture comprises autotrophic cells and substrate, said substrate being configured to promote the growth of the autotrophic cells when the bioreactor is in operation. The method additionally comprises calculating a target range of a concentration of soluble carbon dioxide within the liquid mixture based on, at least in part, on empirical or experimental data, wherein the target range provides for controlled (e.g., increased or enhanced, suppressed or inhibited, etc.) growth of the autotrophic cells when the bioreactor is in use, and comparing the target range of the concentration of soluble carbon dioxide within the liquid mixture to the concentration of soluble carbon dioxide in the liquid mixture. The method comprises adjusting the concentration of soluble carbon dioxide within the liquid mixture by at least one of: (i) modifying a partial pressure of carbon dioxide gas within the headspace, and (ii) modifying the concentration of soluble carbon dioxide within the liquid mixture by delivering a volume of a supplement stream to the liquid mixture. In some embodiments, a concentration of soluble carbon dioxide in the supplement stream is different than the concentration of soluble carbon dioxide of the liquid mixture.

According to some embodiments, the headspace is not in fluid communication with an ambient environment, such that an interior of the bioreactor comprises a closed system. In one embodiment, the bioreactor comprises a cover or some other enclosure device to isolate the liquid mixture and the headspace from the outside (e.g., ambient) environment. In some embodiments, the headspace is in fluid communication with an ambient environment, such that the bioreactor comprises an open system. In one embodiment, the bioreactor comprises a partially open cover or no cover at all.

According to some embodiments, the method additionally includes measuring a temperature of the liquid mixture, wherein the measured temperature of the liquid mixture is used, at least in part, to calculate the target range of a concentration of soluble carbon dioxide. In some embodiments, the method further comprises modifying a temperature of the liquid mixture (e.g., via one or more heating and/or cooling devices or systems) to achieve a target temperature for the liquid mixture, wherein the target temperature assists in enhancing the growth of the autotrophic cells. According to some embodiments, the method further comprises measuring a pH of the liquid mixture, wherein the measured pH is used, at least in part, to calculate the target range of a concentration of soluble carbon dioxide. In some embodiments, the method further comprises modifying a pH of the liquid mixture (e.g., using an injection or fluid delivery system) to achieve a target pH for the liquid mixture, wherein the target pH assists in enhancing the growth of the autotrophic cells.

According to some embodiments, modifying the partial pressure of carbon dioxide gas within the headspace comprises introducing a volume of gas within the headspace of the bioreactor, the volume of gas comprising a concentration of carbon dioxide that is different (e.g., higher or lower) than a concentration of carbon dioxide gas within said headspace. In some embodiments, the gas introduced within the headspace comprises an inert gas having little or no carbon dioxide (e.g., N2, ambient air, etc.).

According to some embodiments, determining the concentration of soluble carbon dioxide within the liquid mixture comprises using a probe, sensor or other measurement device or system. Such a probe or other device or system can be incorporated in the bioreactor system or can be separate and distinct from the bioreactor. In some embodiments, a separate and distinct probe or sensor is in data communication with a control system for the bioreactor. In some embodiments, the probe is inserted or positioned within the liquid mixture to directly determine the concentration of soluble carbon dioxide. In some embodiments, the probe comprises a carbon dioxide probe or sensor. In some embodiments, determining the concentration of soluble carbon dioxide within the liquid mixture comprises calculating the concentration of soluble carbon dioxide using an empirical relationship between soluble carbon dioxide and at least one property of the liquid mixture. In some embodiments, the at least one property of the liquid mixture that is used to calculate the concentration of soluble carbon dioxide comprises pH, total alkalinity, temperature and/or any other property, input or consideration. In some embodiments, measuring the concentration of soluble carbon dioxide with the liquid mixture comprises calculating the concentration of soluble carbon dioxide based on, at least in part, a measured partial pressure of carbon dioxide gas within the headspace, a temperature of the liquid mixture and Henry's constant for carbon dioxide.

According to some embodiments, a supplemental stream of autotrophic cells is configured to be selectively delivered to the bioreactor, wherein the supplemental stream is contained within a supplemental container. In one embodiment, the supplemental stream comprises substrate (e.g., carbon source for cell growth, minerals, nutrients, etc.), wherein the substrate is configured to promote the growth of the autotrophic cells. In some embodiments, the method additionally comprises measuring a concentration of soluble carbon dioxide within the supplemental stream contained within the supplemental container, calculating a target range of a concentration of soluble carbon dioxide within the supplemental stream based on, at least in part, on empirical or experimental data, comparing the target range of the concentration of soluble carbon dioxide within the supplemental liquid to the measured concentration of soluble carbon dioxide in the supplemental liquid, and adjusting the concentration of soluble carbon dioxide within the supplemental liquid by modifying the concentration of carbon dioxide gas within the headspace.

According to some embodiments, the autotrophic cells comprise nitrifying bacteria. In some embodiments, the method further includes adjusting a concentration of ammonium and/or a concentration of nitrite within the liquid mixture to ensure that a growth of the nitrifying bacteria is not nitrogen limited.

According to some embodiments, the autotrophic cells comprise phototrophic microbes. In some embodiments, the autotrophic cells comprise sulfide oxidizing bacteria. In some embodiments, the autotrophic cells comprise precious metal precipitating bacteria. In some embodiments, the method additionally comprises increasing a concentration of hydrogen in the headspace of the bioreactor by injecting hydrogen-rich gas into the headspace in order to promote the growth of the precious metal precipitating bacteria. In one embodiment, the method further includes increasing a dissolved oxygen concentration in the liquid mixture when the autotrophic cells comprise aerobic microbes. In some embodiments, increasing the dissolved oxygen concentration in the liquid mixture comprises injecting an oxygen-laden gas into at least one of said liquid mixture and said headspace.

According to some embodiments, the autotrophic cells comprise Anammox bacteria. In some embodiments, the method further comprises adjusting a concentration of ammonium and/or a concentration of nitrite within the liquid mixture to ensure that a growth of the Anammox bacteria is not nitrogen limited.

According to some embodiments, the method further comprises adjusting an oxidation reduction potential within the liquid mixture by adding a volume of a solution to the liquid mixture, wherein the volume of the solution comprises a concentration of reducing agent that is different than a concentration of reducing agent within the liquid mixture.

According to some embodiments, the autotrophic cells comprise CO2-reducing methanogens. In some embodiments, the autotrophic cells comprise acetogens. In some embodiments, the method additionally includes maintaining a concentration of acetate within the liquid mixture below a threshold level of acetate to ensure proper growth of the acetogens. In some embodiments, the autotrophic cells comprise aceticlastic methanogens. In some embodiments, the method additionally includes maintaining a concentration of acetate within the liquid mixture above a threshold level of acetate to ensure proper growth of the aceticlastic methanogens.

According to some embodiments, the autotrophic cells comprise syntrophic bacteria and CO2-reducing methanogens. In some embodiments, the method further comprises maintaining a concentration of propionate within the liquid mixture above a threshold level of propionate to ensure proper growth of the autotrophic cells. In some embodiments, a mixing intensity within the bioreactor is maintained below a threshold mixing intensity level in order to promote co-localization of the syntrophic bacteria vis-à-vis the CO2-reducing methanogens.

According to some embodiments, the autotrophic cells comprise Syngas-fermenting bacteria. In some embodiments, the method additionally includes adjusting a partial pressure of carbon monoxide within the headspace. In some embodiments, the autotrophic cells comprise dehalogenating bacteria. In one embodiment, the method further comprises maintaining a concentration of halogenated organics within the liquid mixture above a threshold level to ensure proper growth of the dehalogenating bacteria.

According to some embodiments, the autotrophic cells comprise short chain fatty acid methylating microbes. In some embodiments, the method additionally includes maintaining a concentration of substrate short chain fatty acid within the liquid mixture above a threshold level to ensure proper growth of short chain fatty acid methylating microbes. In some embodiments, the autotrophic cells comprise alkane methylating microbes. In one embodiment, the method further comprises maintaining a concentration of substrate alkane within the liquid mixture above a threshold level to ensure proper growth of alkane methylating microbes.

According to some embodiments, the autotrophic cells comprise alcohol methylating bacteria. In some embodiments, the method further comprises maintaining a concentration of substrate alcohol within the liquid mixture above a threshold level to ensure proper growth of alcohol methylating bacteria.

According to some embodiments, it is desirable to intentionally inhibit or suppress growth of autotrophic cells in a bioreactor. For example, it may be desirable in a wastewater treatment train to provide poor growth conditions for autotrophs (e.g., elevated sCO2 concentration) to enhance or promote the operational performance of such a bioreactor (e.g., to selectively promote the growth of other types of cells, e.g., non-autotrophic cells).

According to some embodiments, the bioreactor comprises a stand-alone system for producing autotrophic cells. In some embodiments, the bioreactor is incorporated into an engineered biological system (e.g., wastewater treatment system, sludge/biosolids treatment system, other liquid, gas or solid treatment systems, biochemical production systems and/or the like). In some embodiments, the bioreactor is in fluid communication with a treatment chamber of the wastewater treatment system so that autotrophic cells grown within the bioreactor can be selectively delivered into the treatment chamber.

According to some embodiments, a bioreactor for controlling the growth of autotrophic cells comprises at least one chamber (e.g., tank, container, etc.) for retaining a liquid mixture, an inlet and an outlet in fluid communication with the at least one chamber, wherein the inlet in configured to permit a liquid mixture to enter the bioreactor, and wherein the outlet is configured to permit a liquid mixture to exit the bioreactor and a headspace located above the chamber and the liquid mixture. In some embodiments, the bioreactor comprises at least one probe or sensor configured to determine a concentration of soluble carbon dioxide within a liquid mixture of the bioreactor. The concentration of soluble carbon dioxide can be determined directly (e.g., by using a carbon dioxide sensor or probe) and/or indirectly (e.g., by using an empirical formula that takes into consideration other measured or detected properties of the liquid mixture, headspace and/or the like).

According to some embodiments, the bioreactor further comprises a gas regulation system configured to permit a gas to be selectively moved within the headspace of the bioreactor, wherein the gas regulation system is configured to: (i) alter a concentration of carbon dioxide of the gas moved within the headspace and/or (ii) alter a flowrate of the gas moved within the headspace. The bioreactor can additionally include a control system for regulating a concentration of soluble carbon dioxide within the liquid mixture, wherein the control system is configured to determine a target range of a concentration of soluble carbon dioxide within the liquid mixture based on, at least in part, on empirical or experimental data, wherein the target range provides for controlled (e.g., enhanced or increased growth, suppressed or inhibited growth, etc.) growth of the autotrophic cells when the bioreactor is in use. In some embodiments, the control system is configured to compare the target range of the concentration of soluble carbon dioxide within the liquid mixture to the concentration of soluble carbon dioxide in the liquid mixture. In some embodiments, the control system is configured to adjust the concentration of soluble carbon dioxide within the liquid mixture by at least one of: (i) modifying a partial pressure of carbon dioxide gas within the headspace, and (ii) modifying the concentration of soluble carbon dioxide within the liquid mixture by delivering a volume of a supplement stream to the liquid mixture;

According to some embodiments, the headspace is not in fluid communication with an ambient environment, such that an interior of the bioreactor comprises a closed system. In one embodiment, the bioreactor comprises an upper cover, lid or other enclosure. In some embodiments, the bioreactor comprises an upper enclosure or cover above the liquid mixture. In some embodiments, the headspace is in fluid communication with an ambient environment, such that the bioreactor comprises an open system (e.g., the bioreactor does not include a cover or other enclosure).

According to some embodiments, the bioreactor further comprises at least one additional probe or sensor (e.g., to measure at least one of a temperature, pH, alkalinity, soluble carbon dioxide, etc. of the liquid mixture). In some embodiments, the bioreactor is incorporated into a wastewater treatment system. In some embodiments, the bioreactor comprises an activated sludge treatment tank and/or a digester (e.g., anaerobic digester or system) included in a treatment scheme. In some embodiments, the bioreactor is in fluid communication with an activated sludge treatment tank and/or a digester (e.g., anaerobic digester) included in a treatment scheme.

### Aerobic Autotrophs

In certain circumstances, nitrifying bacteria, cyanobacteria, and biomining microbes are aerobic autotrophs that can be cultivated in a modified bioreactor system that provides optimal sCO₂ for growth.

According to some embodiments, the growth of nitrifying bacteria in wastewater treatment systems can be optimized by the control of the sCO₂ in the aeration basin. In some cases, the cultivation of pure cultures of the nitrifying bacteria may be of interest for seeding biological nitrogen removal systems that treat municipal wastewater or animal waste lagoons.

In certain circumstances, the cultivation of cyanobacteria and other phototrophic, autotrophic microbes has recently generated interest for the production of biodiesel and other biochemicals. Sophisticated bioreactors that utilize natural and artificial light sources can be used for the cultivation of these microbes. In some embodiments, rapid growth of the phototrophic microbes may reduce the capital and/or operation costs associated with the production of the desired endproducts.

In certain circumstances, aerobic, autotrophic microbes are critical for biomining and may be cultivated in a modified batch or chemostat reactor. For biomining, cultivation of adequate biomass levels of autotrophic microbes for sulfide or ferrous iron oxidation and precious metal precipitation may reduce costs associated with biomining. These microbes could be used to seed heap bioleaching piles and bioreactors used to recover precious metals of interest. For biomining microbes, proper aeration, nutrient media, temperature and pH control will be required in addition to sCO₂ control. One predominant sulfide oxidizing bacteria of interest for bioleaching is Acidothiobacillus ferroxidans, which grows aerobically at pH between 1.3 and 4.5 and mesophilic conditions. For thermal heap bioleaching applications, a thermophilic microbe could be cultivated and used to seed the heap pile. Cupriavidus metallidurans strain CH34 is bacteria capable of bioprecipitation of gold from solution as a stress response. Optimization of the pCO₂, and therefore sCO₂, would reduce the doubling time of this gold precipitating microbe.

### Anaerobic Autotrophs

In certain circumstances, several types of anaerobic autotrophs are of interest for wastewater and sludge treatment, industrial use, agricultural, and biomedical applications. Previously, methods were described to improve the specific growth rate of Anammox and methanogens in wastewater and sludge treatment systems. Like the nitrifying bacteria, the cultivation of these microbes would be of interest for seeding biological nitrogen removal systems that are designed for Anammox and anaerobic digesters. In addition, these microbes may also be of interest for seeding wastewater and sludge treatment systems that are designed for animal waste treatment. The methanogens may also be used for bioaugmentation of landfills to promote higher rates of methane production. Beyond wastewater and waste treatment, the methanogens may also be of interest as a new probiotic for human and animal health.

### Probiotics for Hydrogen Reduction in the Large Intestine

In certain circumstances, several diseases or disorders may benefit from the use of a probiotic consisting of one or more autotrophic microbes. Excessive hydrogen production in the large intestine is problematic for human health, since it reduces short-chained fatty acid (SCFA) production and injures colonic mucosa. Hydrogenotrophic methanogens may reduce hydrogen and promote SCFA production by bacteria fermentation. The use of probiotic consisting of methanogens for H₂ reduction has not been considered, but diet modifications towards more complex carbohydrates have been suggested instead. Providing additional carbohydrates to the large intestine does not “solve” the problem of elevated H₂ levels. The elevated levels of H₂ may inhibit fermentation rates by Clostridia and other anaerobic bacteria. Higher rates of fermentation for the production of SCFA may provide additional benefits with respect to butyrate availability for colonic epithelial cells, which provide protection from colonic disease. Another SCFA, propionic acid, may be important for controlling obesity and diabetes type 2. In some cases, the combination of a probiotic consisting of autotrophic microbes and a probiotic consisting of non-pathogenic, fermentative bacteria may ensure high levels of critical SCFA while reducing the risk of infection by pathogenic bacteria. In contrast to the lack of SCFA causing diseases, excessive hydrogen sulfide production in the gut has been implicated in diseases. Ulcerative colitis and chronic fatigue syndrome are thought to be caused by a combination of host genetic factors and sensitivity to reduced sulfur compounds generated by sulfate or sulfur reducing bacteria (SRB) that utilize available H₂ and available sulfur sources. Hydrogen sulfide also increases colonocyte turnover and reduces butyrate metabolism by colonocytes. Low presence of methanogens has been observed in humans with Crohn's disease and ulcerative colitis compared to healthy humans. In each of these diseases, the reduction of pH₂ by a probiotic consisting of autotrophic microbes capable of converting H₂ and CO₂ to acetic acid or CH₄ may offer an effective or preventative treatment for these types of diseases. In addition to human health, probiotics consisting of these autotrophic microbes may also be of interest in Agriculture for the improved health of swine and other non-ruminants.

### Probiotics for Propionate Reduction in the Large Intestine

In certain circumstances, propionic acidemia is a rare genetic disease that results in lower quality of life and deaths due to the inability to breakdown propionic acid. In a healthy human, propionic acid is generated from feed-based amino acid breakdown (˜25%), protein turnover (˜50%), and bacterial fermentation in the large intestine (˜25%). Currently, a modified feed lacking in four amino acids is provided to newborns and children afflicted with propionic acidemia in order to reduce the propionic acid generated in the human body. The reduction in propionic acid due to bacterial fermentation in the large intestine has not been identified as a viable approach. More recently, autism type symptoms have been linked to elevated propionic acid levels in the blood stream due to bacterial fermentations of atypical sugars available in the large intestine for rats. These sugars are present in the large intestine due to low or inactive enzymes for disaccharide and polysaccharide breakdown in the small intestine. The inability to transport these sugars leads to additional sugars available for bacterial fermentation in the large intestine and subsequently more propionic acid. It is unclear whether the pH₂ has caused a shift in the bacteria fermentation endproducts from acetic acid fermentation to propionic acid fermentation, which has been observed and hypothesized in the anaerobic digestion of sewage sludges.

In certain circumstances, autotrophic microbes that oxidize propionate have been identified as syntrophic bacteria and require a CO₂-reducing methanogen to reduce the local pH₂ in order to improve the thermodynamics of this reaction. The slow specific growth rate of these microbes may be attributed to the lack of spatial juxtaposition necessary for the methanogen to reduce the local pH₂ for propionate oxidation by the syntrophic bacteria. In natural and engineered systems, the syntrophic bacteria and CO₂-reducing methanogens have a very tight spatial juxtaposition, which is necessary to reduce the local pH₂. In an upflow anaerobic sludge blanket (UASB) reactors, the spatial relationship of these microbes have been observed in individual granules. The reduction of mixing in anaerobic digesters improved propionate oxidation, which suggested that mixing disrupted the spatial juxtaposition of the syntrophic bacteria and methanogens.

In certain circumstances, for the cultivation of syntrophic bacteria and methanogens, the optimization of sCO₂ through pCO₂ control in the headspace of a batch or chemostat-type bioreactor and the minimization of mixing to prevent the disruption of spatial juxtaposition will be required. The ideal bioreactor system for cultivating this co-culture may be a modified UASB reactor that generates small granules of the autotrophic microbes. For a pure culture or cultures of methanogens, mixing is not inhibitory, so a modified chemostat or batch reactor system with pH₂ and pCO₂ control will be adequate.

In certain circumstances, some H₂-utilizing microbes, such as the CO₂-reducing methanogens and homoacetogenic bacteria, may be attractive as probiotics to outcompete SRB for patients suffering from ulcerative colitis. In addition, higher abundance of H₂-utilizing, autotrophic microbes may also shift the bacteria fermentation of carbohydrates towards acetic acid fermentation, which may be another effective treatment strategy for humans afflicted with Propionic Acidemia. Beyond human health, these probiotics may also be effective in improving the health of animals with similar gastrointestinal tract as humans.

In certain circumstances, syntrophic bacteria may be able to utilize acetate, H₂ and CO₂ to generate propionate. This metabolism, propiogenesis, represents a reversal of propionate oxidation, which may also be of interest for both human and animal health. In this case, the propiogen would be cultivated in a chemostat system that is similar for cultivation of methanogens, except acetate would be included in the nutrient media. The cultivation of propiogens and other SCFA methylating microbes may also be of interest as a probiotic for ruminants, where production of SCFA instead of methane from the available hydrogen may be an effective strategy at reducing the emission of methane, a potent greenhouse gas.

In certain circumstances, the development of these probiotics may also be of interest for environmental applications. The immediate environmental application would be focused on complete anaerobic digestion to biomethane. In anaerobic digesters that are overfed, elevated concentrations of propionic acid are often observed and can lead to a reduction in pH. Extremely high propionic acid concentrations can depress the pH to a point where the anaerobic digester is non-functioning. In these situations, the addition of a probiotic consisting of syntrophic bacteria and CO₂-reducing methanogens, may offer a remedy. The addition of a probiotic consisting of CO₂-reducing methanogens may maintain low pH₂, which may prevent propionic acid fermentations.

In certain circumstances, there is interest in the generation of ethanol and other endproducts, such as acetate, butyrate, lactate, and butanol by Syngas fermenting bacteria, such as Clostridium ljungdahlii, Butyribacterium methlytrophicum, Eubacterium limosum, Clostridium carboxidivorans, Clostridium autoethanogenum and Moorella sp. A cheap supply of Syngas and low-cost bioreactor operation may generate biochemicals that are cost competitive with petroleum derived biochemicals.

In certain circumstances, the cultivation of strict anaerobic bacteria capable of organic dehalogenation may of interest for soil bioremediation. These microbes could be used to seed an above ground bioreactor system that treats polluted groundwater to the surface for biological treatment. If permitted, these microbes could be injected into the subsurface to promote in situ bioremediation.

### Cultivation of Novel Autotrophic Microbes

In certain circumstances, methods for cultivating autotrophic microbes may also allow for the enrichment and isolation of novel microorganisms with biomedical and biotechnological applications. Three types of novel autotrophic microbes are described below.

### SCFA Methylating Microbes

In certain circumstances, autotrophic acetogens are known to have a very flexible metabolism that allows for the utilization of various carbon sources. A thermodynamic evaluation suggests that the methylation of existing SCFA by the use of H₂ and CO₂ is favorable for the generation of longer chained fatty acids. An enrichment and isolation method for autotrophic microbes capable of these reactions would utilize the existing method with a selective medium consisting of the substrate SCFA. For producing SCFA with 3 or more carbons, the headspace pCO₂ could be controlled to ensure optimal growth conditions of the autotroph and the ideal gas of H₂:CO₂ of 3:1 would be injected into the headspace.

### Alkane Methylating Microbes

In certain circumstances, alkane production can result from cow dung and estuarine sediment, suggesting the possibility of microbes capable of methylating alkanes. Similar to SCFA production, the headspace pCO₂ would be controlled to provide optimal growth conditions for the autotroph and the gas injected into the headspace would have a 3:1 of H₂:CO₂ based on the stoichiometry of the overall reaction. The low solubility of the alkanes may require vigorous mixing or aeration to reduce substrate limitation. Longer chain alkanes have higher boiling points, which suggest that the headspace gas could be processed for removal. For ethane, propane, and n-butane, the boiling points are −89° C., −42° C., and 0° C., respectively. A series of bioreactors could be utilized for each subsequent methylation reaction. However, a single bioreactor system with several alkanogens that can provide 2 or more methylation steps would be more ideal. With proper headspace gas processing, the targeted alkane could be selectively removed from the gas stream to prevent endproduct inhibition. For large scale production of alkanes for biofuels, natural gas can be steam reformulated to produce a gas that meets this ideal blend. Electrolysis and photosynthetic bioreactors are other methods for hydrogen production, but require hydrogen separation from oxygen prior to utilization. Further, on-site biological production of longer chain alkanes from the methane in natural gas would be possible by sacrificing some of the natural gas for the production of the gas (3H₂:CO₂) required for the biological reaction. In this manner, propane and butane could be generated solely from methane and may compete on a cost basis with petroleum derived alkanes. A biological reactor system that utilizes methane could also be used to add-value to landfill gas and biogas from anaerobic digesters. In addition, this approach may also be of interest for biomass gasification systems where electricity is normally generated. Instead, the substrate gas (3H₂:CO₂) may be used to generate alkanes from available natural gas.

### Alcohol Methylating Microbes

In certain circumstances, ethanol oxidation can occur by syntrophic bacteria. A thermodynamic evaluation of several novel alcohologenic reactions was conducted. As expected, the methanologenic reaction is unfavorable, but the ethanologenic is favorable for typical environmental and bioreactor conditions. Autotrophic, ethanologenic microbes have not been reported, but warrant further investigation. Of interest are the alcohologenic reactions that methylate existing ethanol and propanol for form propanol and butanol, respectively. Both reactions are favorable with respect to thermodynamics. The capability to producing longer chain alcohols is of interest as a better biofuel alternative to ethanol. Butanol has been receiving more interest as a biofuel, but suffers from fermentation difficulties.

In some embodiments of a bioreactor system, nutrient solution with ethanol would be fed to a bioreactor with a propanologen for the production of propanol. Similar to the alkane bioreactor system, the headspace pCO₂ would be controlled to ensure optimal growth conditions for the propanologen. The ideal gas blend injected into the headspace reflects the stoichiometry of the overall reaction (3H₂:1CO₂). Similar to the alkane bioreactor system, this ideal gas blend would be derived from steam reformation of natural gas rich in methane. A series of bioreactors could be used to generate longer chain alcohols, but a single bioreactor system may be feasible if the various alcohologens have similar nutrient requirements, pH range, etc. In this single bioreactor system, the liquid may be continuously processed by biomass separation (filter or membrane) and the clarified liquid processed for alcohol removal. In contrast to alkanes, the longer chain alcohols have higher boiling points. The liquid could be heated to a temperature where ethanol is boiled, while the propanol and butanol would remain in solution. The ethanol could be recovered and returned to the bioreactor. The liquid with the propanol and butanol could then be heated in a separate reactor to a temperature that exceeds the boiling point of the alcohols. This propanol and butanol rich gas could then be cooled for recovery of the alcohols. This approach becomes more attractive with longer chain alcohols, such as butanol. This type of system would be attractive as an add-on technology for existing ethanol production facilities.

### Cultivation of Methanogens for Bioaugmentation of Anaerobic Digesters

In some embodiments, anaerobic digesters are limited by the slow growth of methanogens, especially at high organic loading rates. For the methanogens in the anaerobic digester, the growth conditions are inhibitory due to the elevated sCO₂ concentration. The bioaugmentation of anaerobic digesters with methanogens would improve the overall reaction rate despite the poor growth conditions. However, the CO₂ in the anaerobic digester biogas can be used as a substrate for the cultivation of CO₂-reducing methanogens in the methanogen bioreactor. The exhaust gas of the methanogen bioreactor would enrich the methane content of the anaerobic digester gas, which can be returned to the headspace of the anaerobic digester. In this configuration, the pCO₂ of the anaerobic digester headspace would be reduced, which would reduce the inhibitory effect of sCO₂ on autotrophs in the anaerobic digester. This represents a dual approach for improving the performance of anaerobic digesters by increasing the methanogen biomass levels and improving the sCO₂ concentration for faster growth rate of the methanogens.

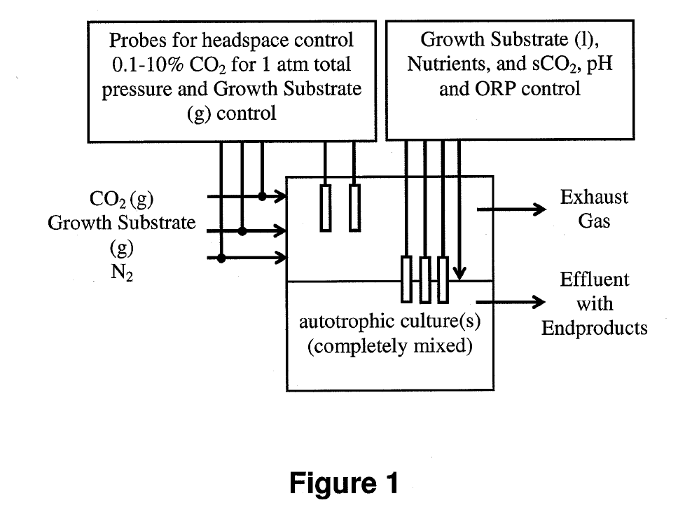

FIG. 1 is a diagram showing one embodiment of a method for controlling the partial pressure of CO₂ (pCO₂) in the headspace of a bioreactor (or area immediately above the upper surface of a liquid contained within the bioreactor) for cultivation of pure or substantially pure autotrophic culture(s). According to some embodiments, measurement of the pCO₂ in the headspace allows for maintaining generally constant and enhanced (e.g., optimal) conditions for autotrophic growth. Injection of inert gases (and/or any other gas that contains no CO₂ or a relatively low concentration of CO₂), such as, for example, N₂, can be used to dilute, and thereby lower, the pCO₂ within or near the bioreactor (e.g., in the headspace of the bioreactor or other system). Alternatively, injection of pure CO₂ or a gas containing a relatively high concentration of CO₂ can increase the pCO₂ in the headspace. In some embodiments, gases necessary for growth, such as H₂, and/or any other component can be injected into the bioreactor (e.g., the headspace, the liquid mixture within the reactor, etc.) to avoid substrate limitation. In some embodiments, the pH and/or oxidation reduction potential (ORP) can be controlled by the use of probes in the media, the addition of pH adjustment and/or ORP adjustment chemicals and/or any other control systems, devices or methods. In some embodiments, prior knowledge of the stoichiometry of the overall biological reaction can facilitate simultaneous injection of some gaseous and/or liquid substrates to avoid or reduce the likelihood of substrate limitation, simplify the system, reduce waste and/or provide one or more other benefits or advantages. Such a configuration can be applied to both suspended growth and fixed-film systems. In yet other embodiments, one or other operating parameters, such as, for example, temperature, other concentrations (e.g., within the liquid mixture, headspace, tec.) and/or the like can be measured and/or controlled in order to achieve a desired growth environment for the microbes being cultivated.

For any of the embodiments disclosed herein, the systems, devices and methods can be applied to any type of reactor or application. For example, the various reactors can be stand-alone reactors (e.g., regardless of whether they are full-scale, lab-scale, etc.) that are used to solely or primarily produce and grow a targeted type of microbe (e.g., autotrophic bacteria). Thus, such stand-alone reactors or systems can be used to grow certain microbes for a customer or other interested end-user. Such customers and/or other end-users can be on or off-site relative to the reactor or system. Alternatively, the reactors or systems can be integrated, directly or indirectly, into one or more other types of systems that need or benefit from the growth and production of certain microbes. For example, is some embodiments, one or more bioreactors configured to grow and cultivate autotrophic bacteria (e.g., methanogens, nitrifying/denitrifying bacteria, Anammox, etc.) can be located within a wastewater treatment plant. Accordingly, the performance of one or more reactors (e.g., treatment tanks, digesters, etc.) or other steps within a treatment train can benefit from receiving a supplemental dose (e.g., continuously or intermittently) of cultivated microbes. For example, an Anammox reactor can be supplemented with Anammox bacteria grown in a separate bioreactor. In other embodiments, the main bioreactor itself (e.g., a mixed liquid tank, reactor, compartment or other chamber of a wastewater treatment system) is controlled according to one or more of the control schemes disclosed herein in order to enhance the operation of such a reactor.

With continued reference to FIG. 1, a bioreactor can include one or more probes, sensors and/or other measuring devices. For example, such components can be positioned within the headspace of the reactor (e.g., and/or region immediately above the liquid mixture) and/or within the liquid mixture itself. In some embodiments, a reactor can include a probe to measure pCO₂ in the headspace, a probe or sensor to detect pH, sCO₂, ORP and/or the any other concentration or property, as desired or required.

According to some embodiments, the sCO₂ of the liquid mixture within the bioreactor is measure using one or more probes. In order to maintain the sCO₂ of the liquid mixture within a desired range (or near a target concentration), the pCO₂ in the headspace (or along the region above the liquid mixture) can be varied. For example, if the sCO₂ of the liquid mixture is relative low (e.g., below a target value or desired range), the pCO₂ of the headspace or the area above the liquid mixture can be increased accordingly. In some embodiments, the pCO₂ above the liquid mixture is increased by providing a gas with a higher concentration of CO₂ and/or by increasing the flowrate at which CO₂-laden gas is passed in the headspace or above the liquid mixture. Alternatively, if the sCO₂ of the liquid mixture is relative high (e.g., above the target or desired range or value), the pCO₂ of the headspace or the area above the liquid mixture can be decreased accordingly. In some embodiments, the pCO₂ above the liquid mixture is decreased by providing a gas with a lower concentration of CO₂ and/or by decreasing the flowrate at which CO₂-laden gas is passed within the headspace or above the liquid mixture.

In other embodiments, the concentration of the sCO₂ of the liquid mixture is regulated by delivering a volume of an adjustment stream (e.g., liquid) into the bioreactor, either in lieu of or in addition to adjusting the pCO₂ above the liquid mixture. Thus, according to some embodiments, such a direct sCO₂ regulation approach relies on the automatic or manual injection or other delivery of a supplemental fluid source (e.g., having a relatively high or low sCO₂) to alter the sCO₂ of the liquid mixture. In some embodiments, such a supplemental fluid source or stream simply dilutes the mixture contained within the bioreactor (e.g., it has a sCO₂ of zero or substantially zero). In other embodiments, a volume of liquid mixture having a relatively high sCO₂ is removed from the bioreactor and replaced with a supplemental fluid having a lower sCO₂. Such control schemes or variations thereof can be incorporated into any of the embodiments disclosed herein.

In any of the embodiments disclosed herein, the probes or other measurement sensor or device (e.g., sCO₂ probe for the liquid mixture) is part of a closed-loop control system with one or more control elements. For example, such a closed-loop system can be configured to regulate (e.g., in real time, on a delayed-time basis, periodically, intermittently, etc.) one or more aspects of the bioreactor, such as, for example, pCO₂, flowrate and/or other characteristics or properties of the gas passed through the headspace or above the surface of the liquid mixture contained within the reactor, the removal of liquid mixture from the reactor, the addition of a supplemental fluid source into the reactor, etc.

Although many of the embodiments disclosed herein are described in the context of a bioreactor or other container that is closed or substantially closed to the atmosphere and the ambient environment, any such embodiments are equally applicable to reactors and/or other systems that are in fluid communication with the ambient surroundings. Thus, for example, in embodiments that disclose the use of a gas (e.g., N₂) to regulate the sCO₂, of the liquid mixture, such a gas can be passed along the top of the liquid mixture without the use of a cover or other member that would otherwise prevent exposure of the gas to the environment.

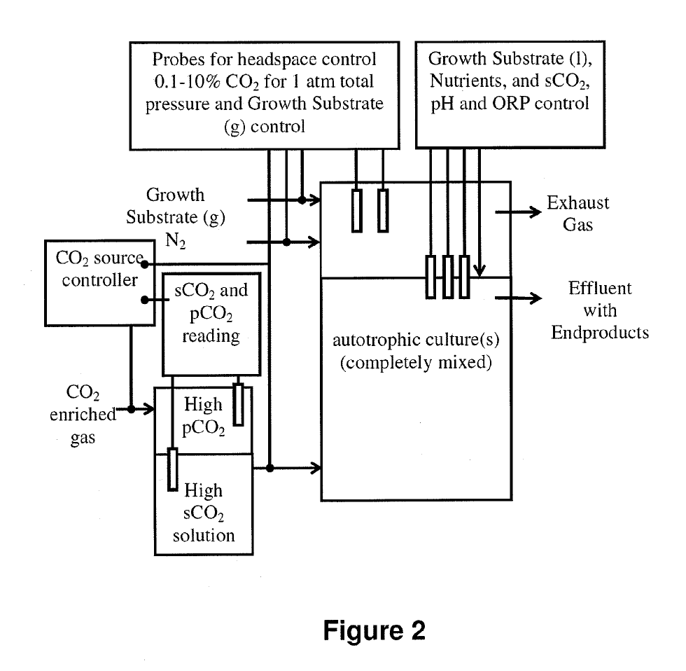

FIG. 2 schematically illustrates another embodiment of a method for controlling pCO₂ in the headspace of a bioreactor for the cultivation of certain microbes (e.g., pure autotrophic cultures). According to some embodiments, as noted above, measurement of the pCO₂ in the headspace allows for maintaining constant and enhanced (e.g., optimal, improved, etc.) conditions for autotrophic growth. Injection of inert gases, such as N₂, can be used to dilute the pCO₂. Injection of a relatively high sCO₂ solution can increase the sCO₂ in the bioreactor and the pCO₂ in the headspace. In some embodiments, the sCO₂ concentration of the high sCO₂ solution can be controlled by pCO₂ in the headspace. A CO₂ source controller can be used to control the concentration of sCO2 in the sCO₂ solution. This controller can also use the measured pCO₂ in the bioreactor headspace to control the transfer of the liquid from the high sCO₂ solution to the bioreactor. In some embodiments, CO₂ or CO₂-enriched gas (e.g., gas having a relatively high CO₂ concentration) can be injected into the headspace of the high sCO₂ solution container to maintain high pCO₂. Gases necessary for growth, such as H₂, can be injected to avoid substrate limitation. The pH and/or ORP can be controlled by the use of probes in the media, the addition of pH adjustment and/or ORP adjustment chemicals and/or any other control systems, devices or methods. In some embodiments, prior knowledge of the stoichiometry of the overall biological reaction occurring within a given bioreactor or other container can facilitate simultaneous injection of some gaseous and/or liquid substrates to avoid or reduce the likelihood of substrate limitation, simplify the system, reduce waste and/or provide one or more other benefits or advantages. Such a configuration can be applied to both suspended growth and fixed-film systems.

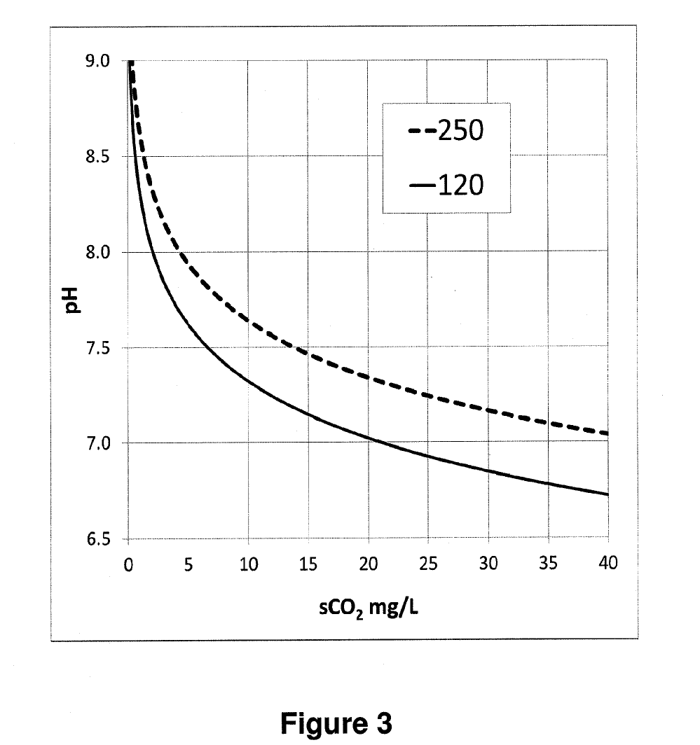

By way of example, for a given wastewater or sludge treatment system where the total alkalinity is known, the pH value is a function of the sCO₂ concentration as shown in FIG. 3. Thus, the sCO₂ concentration within a liquid mixture can be determined (or at least accurately approximated) by measuring or knowing total alkalinity and pH. In the illustrated example, which pertains to wastewater, the total alkalinities are 120 and 250 mg/L as CaCO₃. The sCO₂ concentrations were derived as follows for a given total alkalinity and pH. This same approach using basic water chemistry equations can also be used to derive the pH from the total alkalinity and sCO₂ concentration. The general equation (eq 1) for total alkalinity (eq/L) is provided below as an example. This equation can be expanded to include other significant chemical species that contribute to total alkalinity. This equation can be expressed in terms of the total carbonate species concentration, C_{T,CO3}, and the respective alpha values, which are functions of the pH (eq 2).

TA=[HCO₃⁻]+2*[CO₃²⁻]+[OH⁻]−[H⁺] (eq 1)

TA=C_{T,CO3}*(α₁+2*α₂)+K_{w}/[H+]−[H+] (eq 2)

The above formulae can be used to express C_{T,CO3} as a function of Total Alkalinity (eq 3)

C_{T,CO3}=1/(α₁+2*α₂)*(TA−K_{w}/[H⁺]+[H⁺]) (eq 3)

The alpha values (α) can be calculated directly by first determining E (eq 4).

E=[H⁺]²+[H⁺]*(10^{-6.3}+10^{-6.3}*10^{-10.3}) (eq 4)

α₀=[H⁺]²/E (eq 5)

α₁=[H⁺]*10^{-6.3}/E (eq 6)

α₂=10^{-6.3}*10^{-10.3}/E (eq 7)

Next, the proper alpha values, total alkalinity, K_{w} (10⁻¹⁴), and [H⁺] can be substituted into the C_{T,CO3} equation (eq 3). H₂CO₃*(eq 8) can be calculated using the C_{T,CO3} and α₀.

H₂CO₃*=α₀*C_{T,CO3} mol/L (eq 8)

Finally, the soluble CO₂ can be calculated (eq 9).

CO_{2(aq)}=(H₂CO₃*mol/L)*(44 g/mol)*(1,000 mg/g)mg/L (eq 9)

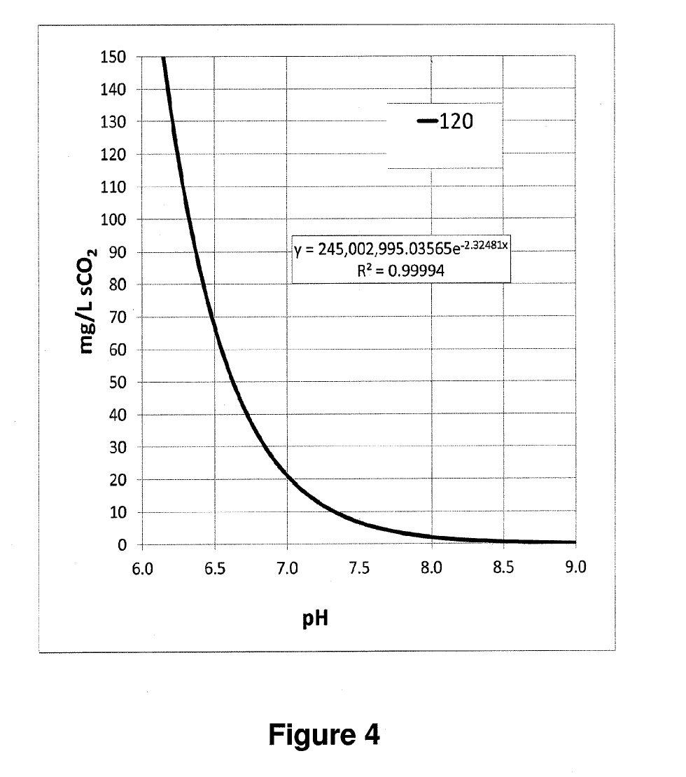

FIG. 4 uses the data from FIG. 3 for wastewater with total alkalinity of 120 mg/L as CaCO₃ to show the sCO₂ concentration as a function of pH. With this type of system curve, the desired sCO₂ concentration for a bioreactor can be maintained by controlling the pH by adjusting the pCO₂ concentration in the headspace by injecting gases with defined pCO₂.

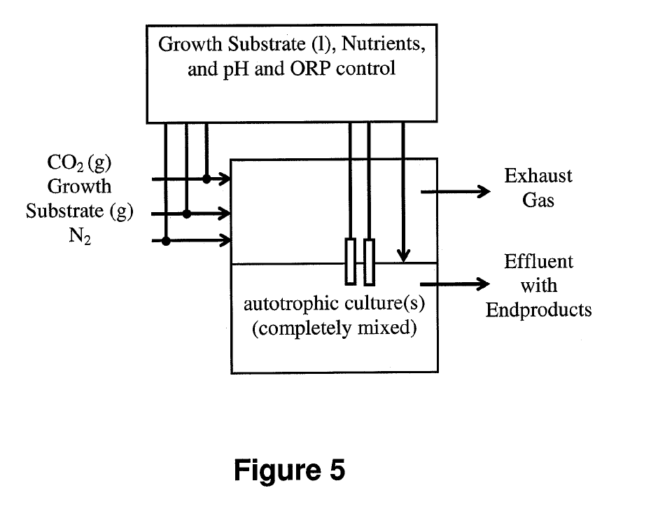

FIG. 5 is a diagram showing one embodiment of a method for controlling sCO₂ of a bioreactor for cultivation of certain microbes (e.g., pure autotrophic cultures) with a defined and constant total alkalinity. According to some embodiments, measurement of the pH in the bioreactor allows for maintaining constant and enhanced (e.g., optimal, improved, etc.) conditions for autotrophic growth. In some arrangements, for a defined total alkalinity, the sCO₂ concentration is a function of pH (see, e.g., the relationship illustrated in FIG. 4). Accordingly, the enhanced or optimal growth conditions for a pure autotrophic culture(s) can be determined for a defined total alkalinity with respect to sCO₂ and pH. In this approach, only a measurement of the pH of the bioreactor is required to control both pH and sCO₂ concentration in the bioreactor. With this determined, the bioreactor can be operated by pH control by measuring the bioreactor pH and adjusting either the pCO₂ in the headspace or the sCO₂ concentration in the bioreactor by gas injection or liquid injection, respectively, as discussed in greater detail herein. For example, injection of inert gases, such as N₂, can be used to dilute the pCO₂ for pH below the optimal pH set point. Injection of pure CO₂ or a gas containing a high concentration of CO₂ can increase the pCO₂ in the headspace and therefore increase the sCO₂ and lower the pH when above the optimal pH set point. Gases and/or other components necessary for growth, such as H₂, substrate, other mineral and nutrients, etc., can be injected to avoid substrate limitation.

According to some embodiments, the ORP can be controlled by the use of a probe in the media, the addition of ORP adjustment chemicals and/or any other control systems, devices or methods. In some embodiments, prior knowledge of the stoichiometry of the overall biological reaction can facilitate simultaneous injection of some gaseous and/or liquid substrates to avoid or reduce the likelihood of substrate limitation, simplify the system, reduce waste and/or provide one or more other benefits or advantages. Such a configuration can be applied to both suspended growth and fixed-film systems.

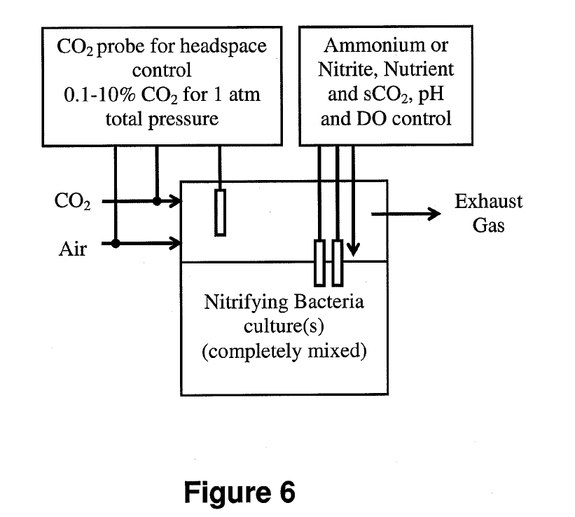

FIG. 6 is a schematic diagram showing one embodiment of a modified reactor configuration for the cultivation of nitrifying bacteria that can grow relatively rapidly with enhanced (e.g., improved, optimal, etc.) sCO₂ by control of the headspace pCO₂ concentration. A dissolved oxygen (DO) probe can be used to ensure that oxygen is non-limiting. In other words, an oxygen probe or other sensor can be used to regulate (e.g., either automatically, as part of a larger control scheme, or manually) an oxygen concentration within a desired range. In other embodiments, one or more other types of probes, measuring devices and/or other instruments can be incorporated into a system, either in lieu of or in addition to a DO probe. In addition, in some embodiments, aeration of the reactor contents using the headspace gas can help prevent or reduce the likelihood of oxygen limitation and can help ensure adequate sCO₂. In some arrangements, pure oxygen is used instead of ambient air to help prevent and/or reduce the likelihood of oxygen limitation. The reactor can be operated in batch or continuous flow mode, as desired or required for a particular application or use.

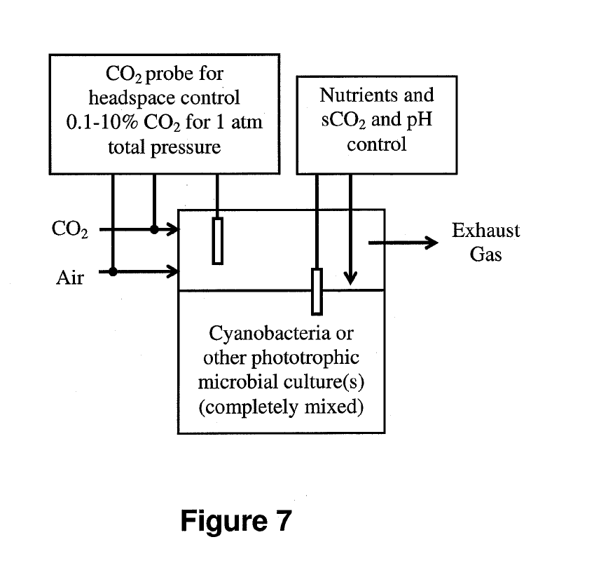

FIG. 7 is a diagram schematically showing one embodiment of a modified reactor configuration for the cultivation of phototrophic, autotrophic microbes, such as, for example, Cyanobacteria, that grow relatively rapidly with optimal or otherwise enhanced sCO₂ by control of the headspace pCO₂ concentration. In some embodiments, the reactor is exposed to adequate light to facilitate growth of the phototrophic microbes. The reactor can be operated in batch or continuous flow mode.

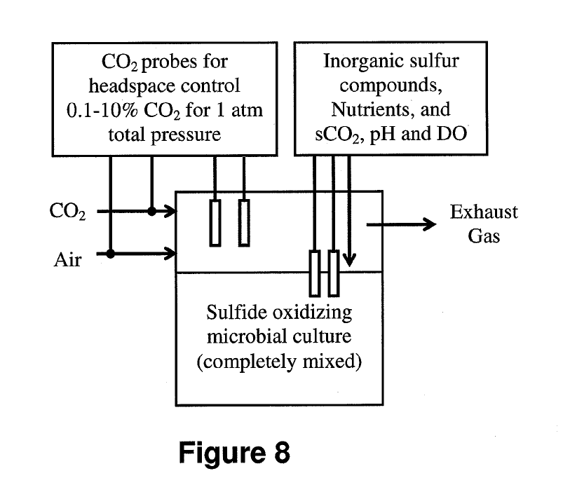

FIG. 8 is a diagram schematically showing one embodiment of a modified reactor configuration for the cultivation of sulfide oxidizing microbes that grow rapidly with optimal sCO₂ by control of the headspace pCO₂ concentration. A dissolved oxygen (DO) probe can be used to ensure that oxygen is non-limiting or otherwise to maintain a desired DO concentration. In addition, aeration of the reactor contents using the headspace gas can prevent oxygen limitation and help ensure adequate sCO₂. In some arrangements, pure oxygen may be used instead of (or in addition to) air to prevent oxygen limitation. The reactor can be operated in batch or continuous flow mode.

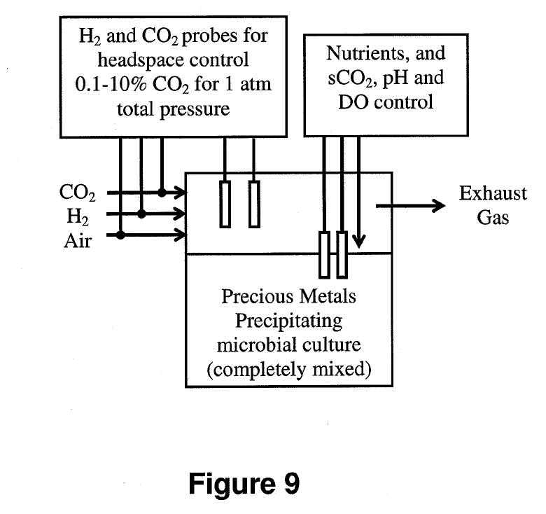

FIG. 9 is a diagram schematically showing one embodiment of a modified reactor configuration for the cultivation of metals precipitating microbes that grow rapidly with enhanced (e.g., improved, optimal, etc.) sCO₂ by control of the headspace pCO₂ concentration. A dissolved oxygen (DO) probe can be used to ensure that oxygen is non-limiting. In addition, aeration of the reactor contents using the headspace gas will prevent or help ensure against oxygen limitation and/or will help ensure that adequate sCO₂ is present. In some arrangements, pure oxygen may be used instead of or in addition to air to help prevent oxygen limitation. The reactor can be operated in batch or continuous flow mode.

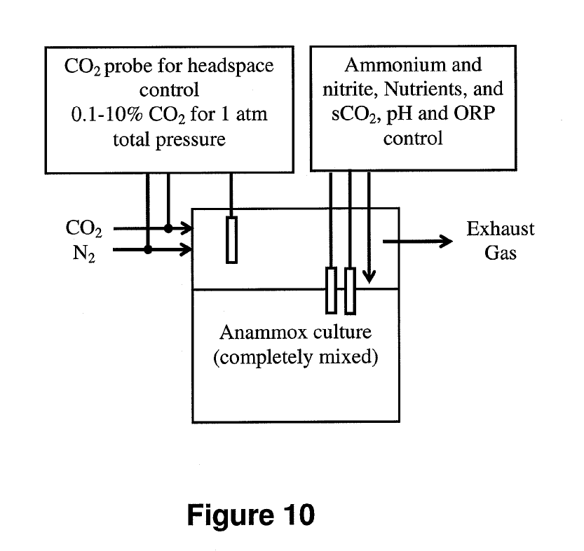

FIG. 10 is a diagram schematically showing one embodiment of a modified reactor configuration for the cultivation of Anammox bacteria that grow relatively rapidly with enhanced (e.g., improved, optimal, etc.) sCO₂ by control of the headspace pCO₂ concentration. An ORP probe in combination with a reducing agent transfer system can be used to ensure that reducing conditions are conducive for growth. The reactor can be operated in batch or continuous flow mode.

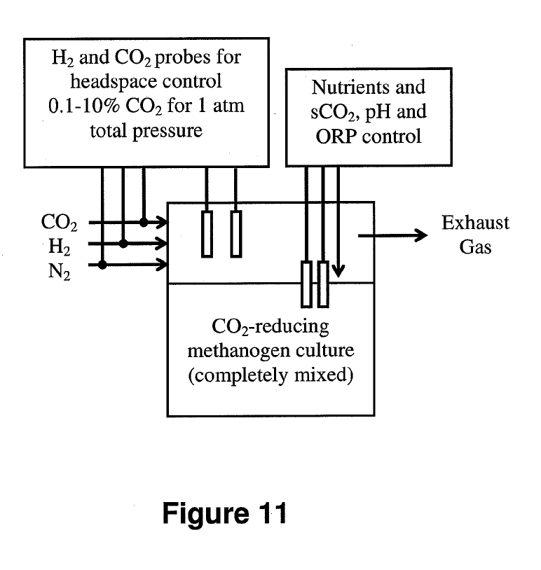

FIG. 11 is a diagram schematically illustrating one embodiment of a modified reactor configuration for the cultivation of CO₂-reducing methanogens with optimal or enhanced sCO₂ by control of the headspace pCO₂ concentration. An ORP probe in combination with a reducing agent transfer system can be used to ensure that reducing conditions are conducive for growth.

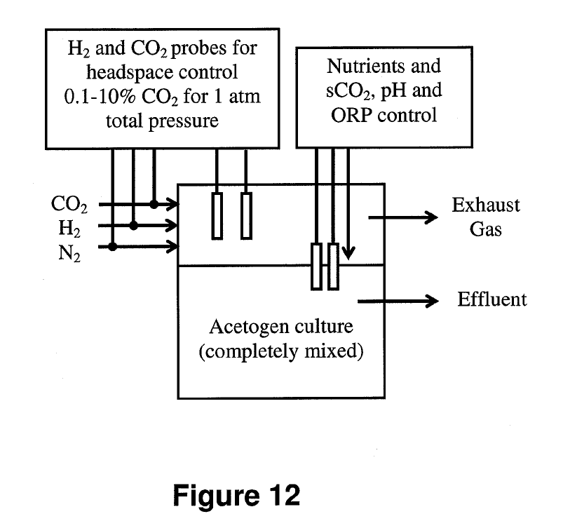

FIG. 12 is a diagram schematically showing one embodiment of a modified reactor configuration for the cultivation of acetogens with optimal or enhanced sCO₂ by control of the headspace pCO₂ concentration. An ORP probe in combination with a reducing agent transfer system can be used to ensure that reducing conditions are conducive for growth.

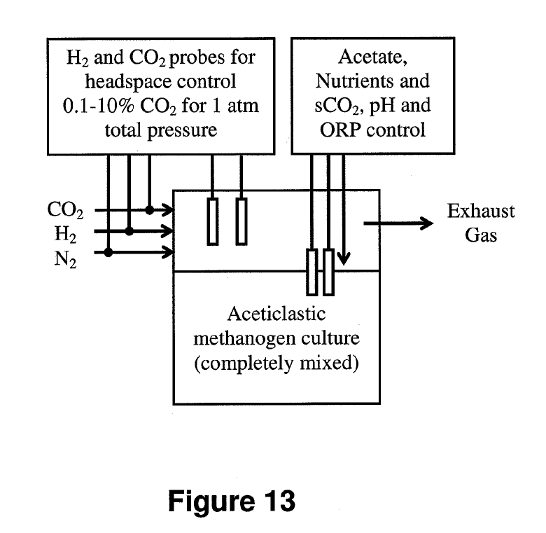

FIG. 13 is a diagram schematically showing one embodiment of a modified reactor configuration for the cultivation of aceticlastic methanogens with optimal or enhanced sCO₂ by control of the headspace pCO₂ concentration. An ORP probe in combination with a reducing agent transfer system can be used to ensure that reducing conditions are conducive for growth.

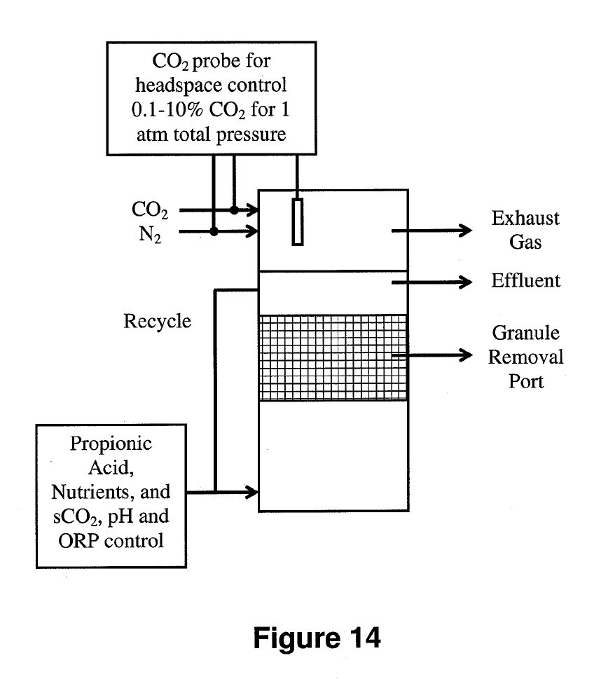

FIG. 14 is a diagram showing one embodiment of a modified UASB reactor configuration for the cultivation of a co-culture of syntrophic bacteria and CO₂-reducing methanogens with optimal or enhanced sCO₂ by control of the headspace pCO₂ concentration. As for any other embodiments of a bioreactor disclosed herein, an ORP probe in combination with a reducing agent transfer system can be used to help ensure that reducing conditions are conducive for growth. The microbes are in the granules within the sludge blanket (shaded) suspended within the liquid media.

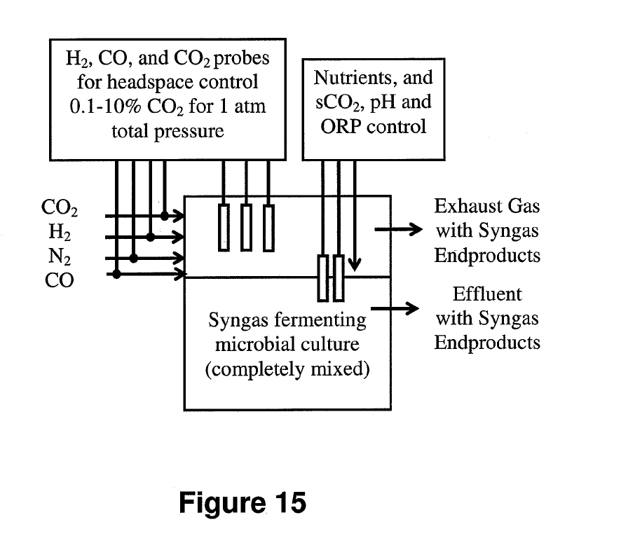

FIG. 15 is a diagram schematically showing one embodiment of a modified reactor configuration for the cultivation of autotrophic microbes that ferment Syngas that grow rapidly with optimal or enhanced sCO₂ by control of the headspace pCO₂ concentration. In such an embodiment, Syngas is added to the bioreactor and the microbes use, inter alia, H₂, CO, and CO₂ to generate gaseous and soluble endproducts. The reactor can be operated in batch or continuous flow mode.

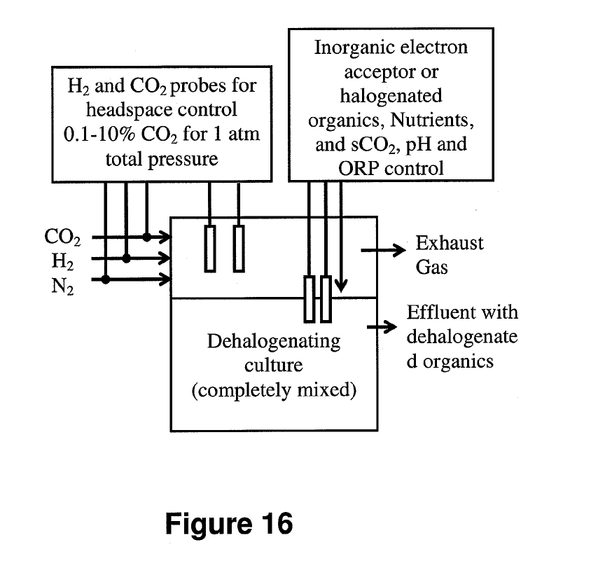

FIG. 16 is a diagram showing one embodiment of a modified reactor configuration for the cultivation of autotrophic, dehalogenating bacteria that grow rapidly with optimal or enhanced sCO₂ by control of the headspace pCO₂ concentration. Inorganic electron acceptor or halogenated organics are added to the bioreactor and the microbes use H₂ and CO₂ to generate gaseous or soluble endproducts. The reactor can be operated in batch or continuous flow mode.

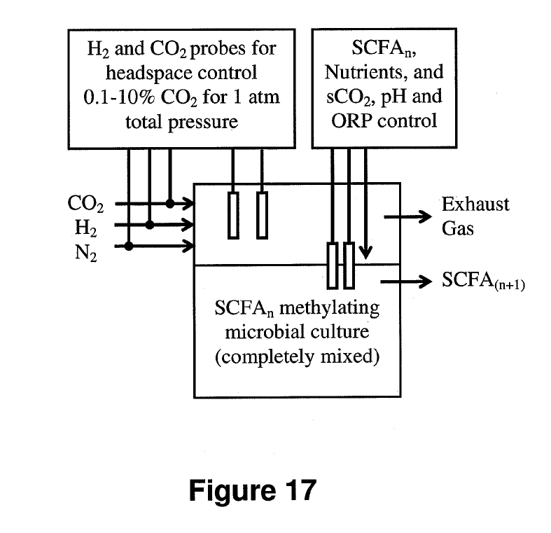

FIG. 17 is a diagram schematically showing one embodiment of a modified reactor configuration for the cultivation of autotrophic microbes that generate SCFA₍ₙ₊₁₎ by methylation that grow rapidly with optimal or enhanced sCO₂ by control of the headspace pCO₂ concentration. SCFAₙ, where n=# of carbon atoms, is added to the bioreactor and the microbes use H₂ and CO₂ to methylate the SCFAₙ and generate SCFA₍ₙ₊₁₎. The reactor can be operated in batch or continuous flow mode.

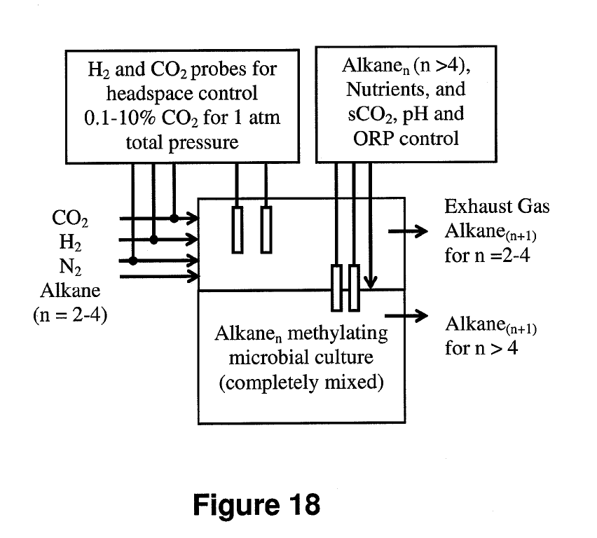

FIG. 18 is a diagram schematically showing one embodiment of a modified reactor configuration for the cultivation of autotrophic microbes that generate Alkane₍ₙ₊₁₎ by methylation that grow rapidly with optimal or enhanced sCO₂ by control of the headspace pCO₂ concentration. Alkaneₙ, where n=# of carbon atoms, is added to the bioreactor as a gas or liquid and the microbes use H₂ and CO₂ to methylate the Alkaneₙ and generate Alkane₍ₙ₊₁₎. The reactor can be operated in batch or continuous flow mode. In some embodiments, for ethane, propane, and butane, the exhaust gas is processed for recovery of alkanes. In some embodiments, for pentane (b.p.=36° C.) or alkanes with greater # of carbon atoms, the filtered liquid is processed for alkane recovery.

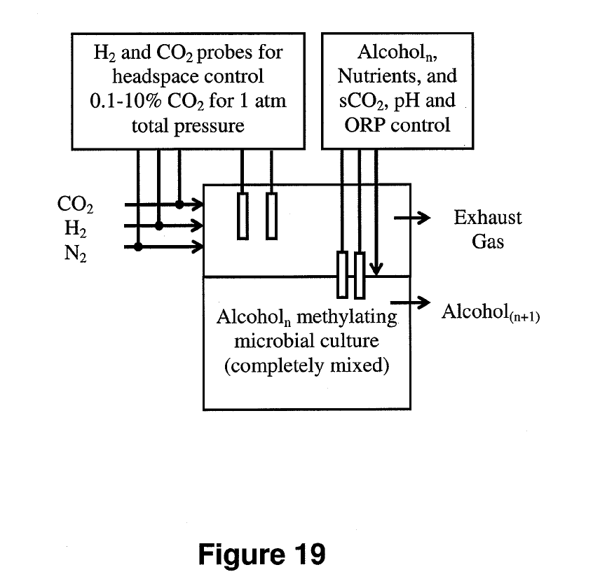

FIG. 19 is a diagram schematically showing one embodiment of a modified reactor configuration for the cultivation of autotrophic microbes that generate Alcohol₍ₙ₊₁₎ by methylation that grow rapidly with optimal or enhanced sCO₂ by control of the headspace pCO₂ concentration. Alcoholₙ, where n=# of carbon atoms, is added to the bioreactor as a liquid and the microbes use H₂ and CO₂ to methylate the Alcoholₙ and generate Alcohol₍ₙ₊₁₎. The reactor can be operated in batch or continuous flow mode. According to some embodiments, the filtered liquid is processed for alcohol recovery.

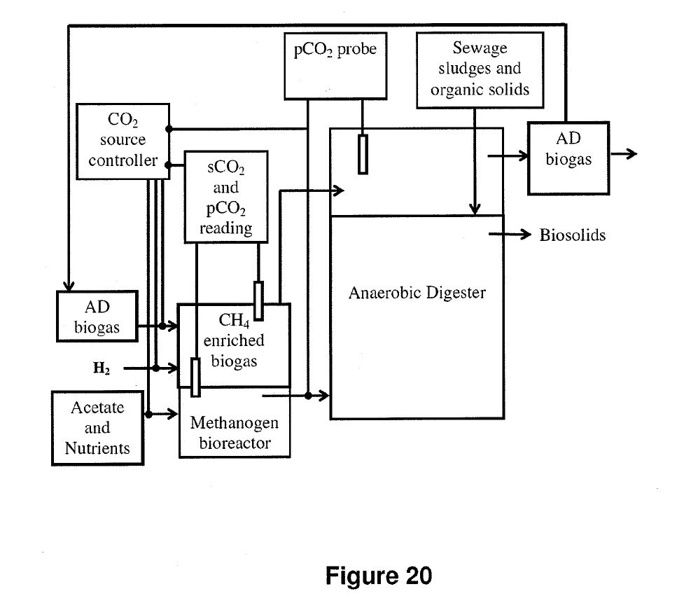

FIG. 20 is a diagram schematically showing one embodiment of a modified reactor configuration for the cultivation of methanogens with optimal or enhanced sCO₂ by control of the headspace pCO₂ concentration. The reactor can be operated in batch or continuous flow mode. According to some embodiments, the effluent from the methanogen bioreactor is fed to the anaerobic digester. A fraction of the anaerobic digester biogas can be transferred to the headspace of the methanogen bioreactor in order to meet the CO₂ demand of the methanogens. The exhaust of the methanogen bioreactor is transferred to the headspace of the anaerobic digester, which improves the growth of the autotrophic microbes in the anaerobic digester. A CO₂ source controller is used to control the headspace CO₂ concentration and sCO₂ concentration in the methanogen bioreactor. In addition, this CO₂ controller also controls the influent flow rates of anaerobic digester biogas, H₂, and Acetate and Nutrient solution; the transfer flow rates of the effluent of the methanogen bioreactor and exhaust (CH₄ enriched biogas) to the anaerobic digester headspace.

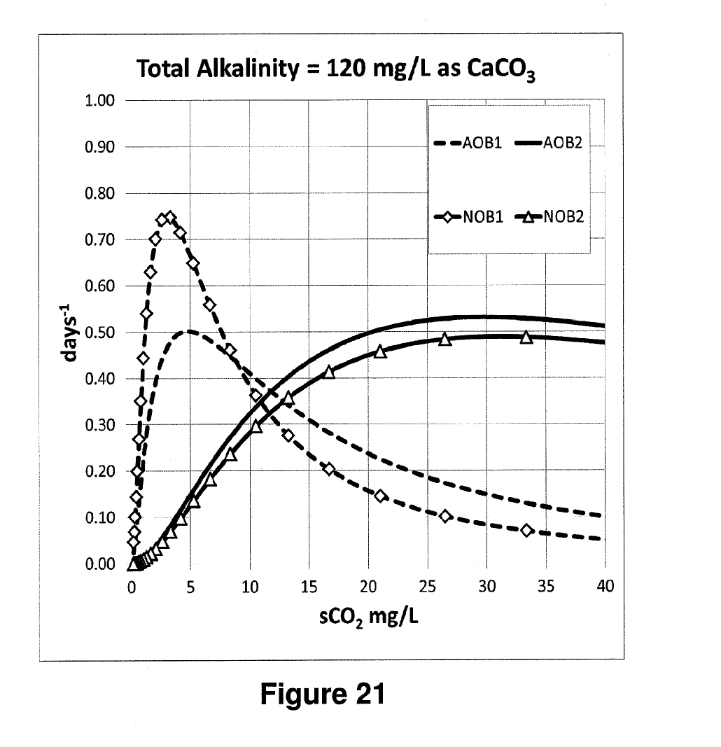

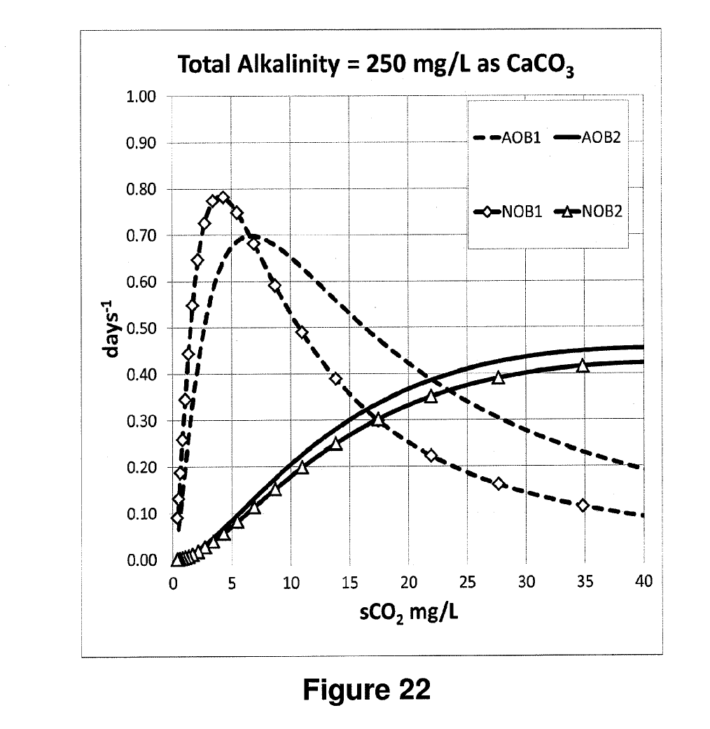

According to some embodiments, using estimated growth parameters of certain microbes (e.g., nitrifying bacteria), the specific growth rate can be computed as a function of sCO₂ concentration and the associated pH for a given total alkalinity (FIG. 3) as shown, e.g., in FIGS. 21 and 22. By way of example, the sCO₂ concentration in aeration basins of wastewater treatment systems can be in the range of 10 to 25 mg/L CO₂, with higher concentrations found near the inlet. Aeration basins with a large range of sCO₂ can support the growth of all nitrifying bacteria, but may indicate inefficient nitrification rates. The presence of both pairs of nitrifiers has been interpreted as a sign of diversity, which is to be promoted. However, such an analysis suggests that the presence of both pairs of nitrifiers is due to sCO₂ concentration variability, which causes lower nitrification rates. In some embodiments, one simple remedy for this situation is to blend basin exhaust air containing elevated CO₂ into the feed of the aeration system in the basin exhibiting low sCO₂ concentration. Another possible solution includes recycling the basin exhaust air directly into sections of the basin exhibiting low sCO₂ concentration. Accordingly, in some embodiments, the basin can select for one dominant nitrifying bacteria pair for higher nitrification rates. In some embodiments, such an approach can also be beneficial for daily or seasonal changes in influent total alkalinity due to dilution. For high total alkalinity (FIG. 22), increased aeration rates may reduce the sCO₂ concentration for higher rates of nitrification.

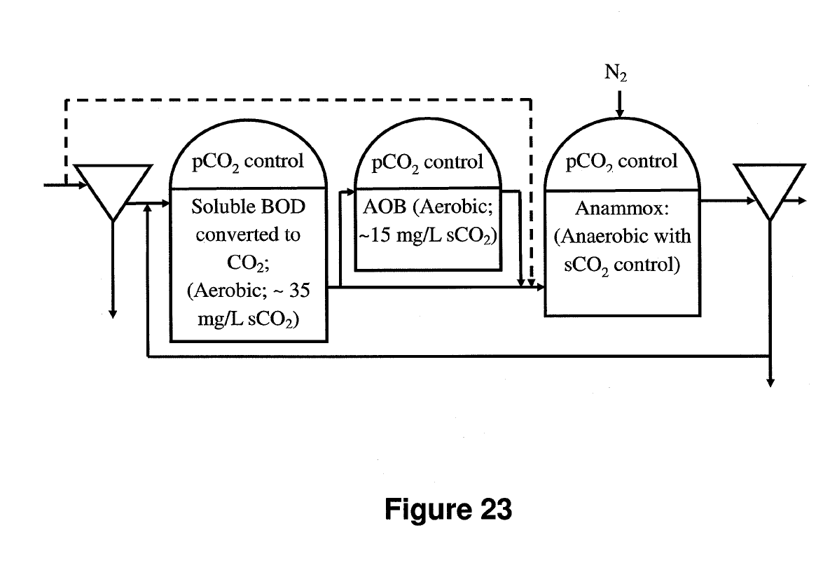

With the ability to measure the total alkalinity and pH of a wastewater, a wastewater treatment plant with relatively higher alkalinity can be modified for efficient biological nitrogen removal, biological nutrient removal, and biomethane generation and biological nutrient removal as shown in FIGS. 23-25. All three configurations have a common biological nitrogen removal treatment train that features a series of basins that select for AOB and Anammox bacteria. In some embodiments, operation at a low SRT presents a strong selective pressure against slow growing nitrite oxidizing bacteria when the initial aeration basin is operated at an elevated sCO₂ concentration of about 35 mg/L, for example. Several methods can be used to ensure elevated sCO₂ concentration, including lift station retrofit, recycle of aeration basin exhaust, and recycle of gas or diesel generator exhaust. In some embodiments, the second aeration basin accepts about half of the effluent from the first aeration basin and is operated at a low sCO₂ of about 15 mg/L (e.g., 3-20 mg/L, 3-5, 5-10, 10-15, 15-20 mg/L, etc.) in order to provide optimal growth conditions for the ammonia oxidizing bacteria as shown in FIG. 22. In some arrangements, intense aeration is required to strip the CO₂ from the influent wastewater but sCO₂ control may still be necessary to select for the AOB. The effluent from the second aeration basin is combined with the remaining effluent from the first aeration basin and is transferred to an anaerobic basin that is operated to promote the growth of the Anammox bacteria. The dashed line from the influent wastewater to the anaerobic basin represents a low flow rate that may be necessary to ensure strict anaerobic conditions. The anaerobic basin is covered with pCO₂ control made possible by the use of N₂ gas (and/or another type of inert gas). With total alkalinity and pH measurements, the sCO₂ can be controlled in each basin.

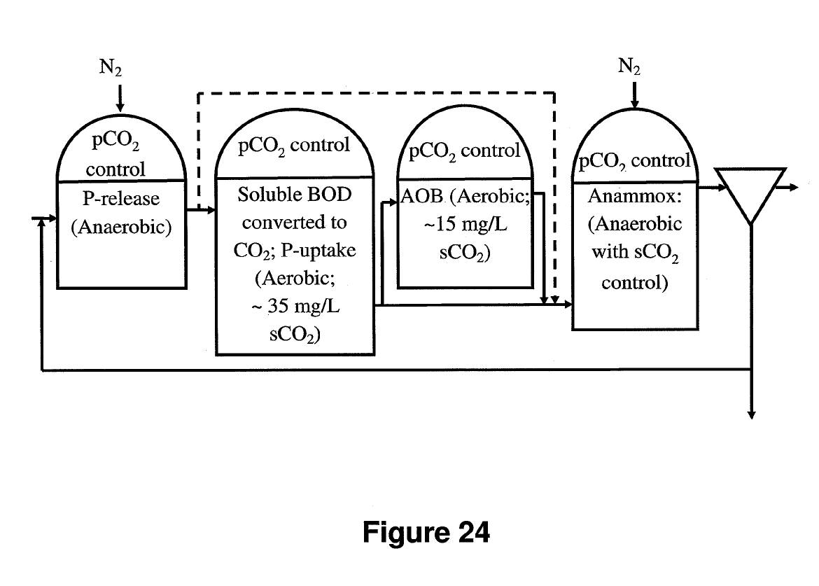

If phosphorus removal is also desired, the wastewater treatment plant schematically shown in FIG. 23 can be further modified as schematically shown in FIG. 24. In this configuration, the anaerobic basin can be covered to allow for sCO₂ control. In some instances, the influent may not be clarified, which will provide strict anaerobic conditions. This configuration allows for the P-release by phosphorus accumulating organisms (PAO), which occurs under strict anaerobic conditions and the availability of volatile fatty acids from the fermentation of primary solids and soluble BOD. In some embodiments, operation at an elevated sCO₂ of about 35 mg/L in the anaerobic basin also prepares the wastewater for treatment in the first aeration basin. Nitrogen gas may be used to control the pCO₂ in the headspace of the anaerobic basin. In this configuration, a mechanical mixer or aeration system that recycles the headspace gas could be used to control the sCO₂ concentration.

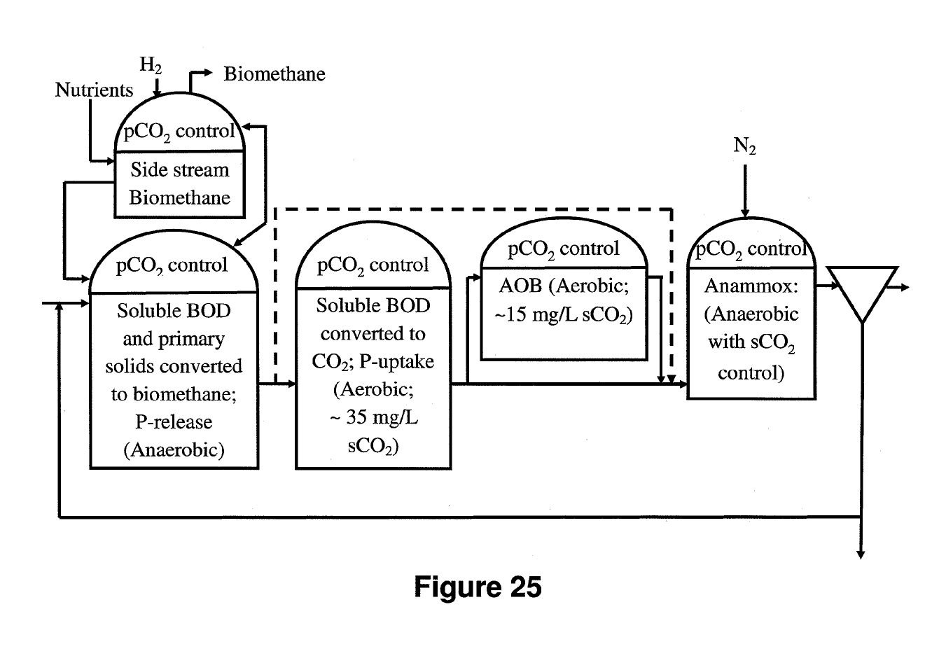

In some cases the anaerobic basin may be further modified to generate biomethane as schematically shown in FIG. 25. A side stream biomethane reactor (FIG. 20) can be used to bioaugment the anaerobic basin with aceticlastic methanogens.

The autotrophic microorganisms are found in the Bacteria and Archaea branches of the Tree of Life. Several types of autotrophic microbes including the nitrifying bacteria, Anammox bacteria, sulfate reducing bacteria (SRB), acetogens, dehalogenating bacteria, sulfur and sulfide oxidizing microbes, metal precipitating microbes, methanogens, and others have value for environmental remediation, but have limited application due to their slow specific growth rate or doubling time that is often reported on the order of days.

In some embodiments, pure cultures of autotrophic bacteria and archaea can be cultivated in bioreactors that control the pCO₂ in order to provide the optimal or enhanced sCO₂ for growth. Rapid growth of autotrophic microbes advantageously reduces the capital and operating costs associated with producing these pure cultures for biomedical, biotechnological and/or other applications.

### Andrew's Equation

Andrew's equation describes the relationship between specific growth rate of autotrophic microbes and dissolved carbon dioxide. Three parameters are used to define Andrew's equation for anaerobic autotrophs: μₘₐₓ, K_{s,CO2}, and K_{i,CO2}, where μₘₐₓ is the maximum specific growth rate, h⁻¹; K_{s,CO2} is the saturation constant for CO₂, mg/L; and K_{i,CO2} is the inhibition constant for CO₂, mg/L. [CO2] is the concentration of CO₂. The specific growth rate (μ_{obs}) is reduced by the decay coefficient (b or k_{d}). The parameters μₘₐₓ, Kₛ, Kᵢ, and b are estimated to best fit the observed specific growth rates.

$$μobs = μmax \times \frac{[ CO 2 ]}{[ CO 2 ] + Ks , CO 2 + \frac{{[ CO 2 ]}^{2}}{Ki , CO 2}} - b \qquad ( eq 10 )$$

Microbes are generally sensitive to pH, and the Andrew's equation can be combined with a Monod term for pH that will provide method of describing the specific growth rate.

$$μobs = μmax \times \frac{[ CO 2 ]}{[ CO 2 ] + Ks , CO 2 + \frac{{[ CO 2 ]}^{2}}{Ki , CO 2}} \times \frac{1}{( 1 + \frac{[ H + ]}{K 1} + \frac{K 2}{[ H + ]} )} - b \qquad ( eq 11 )$$

In the Monod term for pH, [H⁺] represents the proton concentration and K₁ and K₂ represent the pH factor range limits for growth. K₁ represents the lower pH limit and K₂ represents the upper pH limit. For example, if the pH factor is set for a range of pH between about 6 and about 8 then K₁ would be 10⁻⁶ and K₂ would be 10⁻⁸. Methanogens have been observed to grow at a very broad pH range of between a pH of about 3 to about 9. However, the methanogens in the anaerobic digesters and in the animal or human digestive system have a generally neutral pH range of about 6 to about 8. Beyond sCO₂ and pH, in some embodiments, growth substrates can also be included as a Monod term. However, the concentrations of the growth substrates are typically maintained at values that ensure non-limitation, which means that the Monod term has a value of approximately 1 (e.g., about 0.8, 0.9, 1.0, 1.1, 1.2, values between the foregoing, etc.).

In some embodiments relating to autotrophs, the dissolved CO₂ or soluble CO₂ (sCO₂) is typically not optimal or enhanced with respect to specific growth rate, which can limit biomedical and biotechnology applications utilizing these microbes. According to some arrangements, for the cultivation of pure cultures of autotrophic microbes, a bioreactor requires simple modification to ensure control of the pCO₂ in the headspace, which directly controls the sCO₂ in the liquid media (FIG. 1). With reference to the schematic embodiment illustrated in FIG. 1, a pCO₂ probe is used to measure the headspace pCO₂, and this measurement is used to regulate the concentration of CO₂ in the headspace. For example, in some embodiments, CO₂ is added to the headspace to increase pCO₂. Depending on the measured concentration by the probe, however, an inert gas, such as, for example, N₂, is added to the headspace to effectively dilute CO₂ and thus reduce the pCO₂ concentration. A simple pCO₂ controller may be used to set the target pCO₂ in the headspace. Thus, control of pCO₂ can be advantageously accomplished automatically according to a particular control scheme (e.g., feedback loop control). In alternative embodiments, however, control of the pCO₂ is accomplished manually. Some controllers utilize a probe that measures % CO₂, which can be utilized when the headspace pressure is also measured. In some embodiments, the sCO₂ in the bioreactor liquid media is directly related to the pCO₂ or the total headspace pressure and % CO₂ in the headspace. Either system could be utilized to directly control the sCO₂ to ensure that the optimal or enhanced sCO₂ is provided to the pure culture of autotrophic microbe being cultivated in the bioreactor. In the figure provided, the target range of 0.1-10% CO₂ for headspace of 1 atm will provide a sCO₂ concentration range of 1-114 mg/L at 35° C., which are optimal or enhanced growth conditions for mesophilic autotrophs. Lower temperature cultivation will require a pCO₂ range that is narrower due to the temperature sensitivity of Henry's constant for CO₂. For example, cultivation at 25° C. will require a pCO₂ range of 0.07-7.7% for the same sCO₂ concentration range of 1-114 mg/L. Similarly, thermophilic autotrophs will require a broader pCO₂ range. For example, cultivation at 65° C. will require a pCO₂ range of 0.17-19.8%. In some embodiments, a slightly elevated headspace pressure will assist in preventing or reducing the likelihood of leaks, especially for anaerobic operation. In some embodiments, the gases that are injected into the headspace of the bioreactor are filter sterilized to prevent or reduce biological and/or other contamination (e.g., using a 0.2 μm filter).

For anaerobic operation, according to some embodiments, oxygen is removed from the gases by passing the gas through an oxygen scavenger system prior to injection into the headspace. Liquid growth substrates, nutrient solutions, pH adjustment solutions and/or the like are preferably sterilized prior to use, and proper anaerobic technique utilized, if necessary. In addition to CO₂, growth substrate in the gaseous form (ex. H₂) or liquid form can be added to the headspace or bioreactor media, respectively. Probes in the headspace or liquid media can be used to ensure that non-limiting concentrations of growth substrate are provided to the microbe. In addition, pH control through the addition of buffer and/or strong acids or bases will be possible through the use of an automated system that includes a pH probe. Nutrients can also be added to the media. Temperature control systems can be incorporated into the system to help ensure that the bioreactor is operated at the optimal or preferred temperature or temperature range in order to promote microbial growth.

In some embodiments, such bioreactors are operated as suspended growth systems or fixed film systems. Also, the bioreactor can be operated as a continuously fed batch reactor (i.e., chemostat) or a fed batch reactor. Such bioreactor configurations can also be used for enriching for autotrophic microbes of interest by providing the appropriate selective media. Identical or similar systems could also be used to modify an incubator to allow for isolation of pure cultures on agar plate surfaces. Several examples for cultivating autotrophic microbes are provided, which utilize this reactor configuration.

### Cultivation of Aerobic Autotrophic Microbes

In some embodiments, nitrifying bacteria can grow faster with optimal or enhanced pH and/or sCO₂ concentrations. Bioreactors operated in batch mode can be used to enrich for both ammonium oxidizing bacteria (AOB) and nitrite oxidizing bacteria (NOB), as desired or required. In other embodiments, the headspace pCO₂ of a bioreactor is controlled by the method that is generally described herein with reference to the schematic of FIG. 1. This embodiment is one preferred embodiment for autotrophic microbes that only use CO₂ as an anabolic carbon source. For high concentrations of biomass and/or CO₂ being used as a catabolic carbon source, an alternative embodiment may be more suitable as shown in FIG. 2. In this embodiment, a high sCO₂ solution is injected into the bioreactor in order to maintain optimal or enhanced sCO₂ concentration. The embodiment shown in FIG. 2 may also be combined with the embodiment shown in FIG. 1.

In some embodiments, a bioreactor with a defined and constant total alkalinity requires simple modification to ensure control of the sCO₂ concentration via the pH (FIG. 5). With reference to the schematic embodiment illustrated in FIG. 5, a pH probe is used to measure the bioreactor pH, and this measurement is used to regulate the concentration of sCO₂ in the bioreactor. With a defined and constant total alkalinity, the sCO₂ concentration is a function of the pH as shown in FIG. 4. The enhanced (e.g., optimal, improved, etc.) growth conditions for the autotrophic microbe can be determined for a combination of the sCO₂ concentration and associated pH value for a defined total alkalinity. This pH value is the set point for the bioreactor operation with respect to sCO₂ concentration. If the measured pH value is less than the pH set point, then the sCO₂ is too high and it can be reduced by either injecting CO₂-free gas into the headspace of the bioreactor or transferring a liquid with low sCO₂ concentration into the bioreactor. If the measured pH value is greater than the pH set point, then the sCO₂ is too low and it can be increased by either injecting CO₂-enriched gas into the headspace of the bioreactor or transferring a liquid with high sCO₂ concentration into the bioreactor. The embodiment shown in FIG. 5 may also be combined with the embodiments shown in FIGS. 1 and 2, and/or any other embodiments disclosed herein.

With reference to FIG. 6, a modified reactor system can be used for cultivation of either nitrifying bacteria (i.e., AOB or NOB) or both. By way of example, if pCO₂ is too high, then air and/or other gases can be injected into the headspace of the system to reduce the pCO₂. In some embodiments, for cultivation of both AOB and NOB, ammonium is added to the reactor to ensure non-limiting conditions. For cultivation of NOB, nitrite can be provided to the reactor. According to some arrangements, the use of a fixed-film system or membrane bioreactor configuration allows for the dilution of the nitrate concentration in the bioreactor to prevent or reduce the likelihood of inhibition. In some embodiments, settling of the biomass and decant of the supernatant can also accomplish the same goal.

Phototrophic, autotrophic microbes, such as Cyanobacteria, can be cultivated in a modified bioreactor, as shown in FIG. 7. Natural or artificial light sources can be used to advantageously promote growth of the phototrophs in the bioreactor. Air and/or other fluids (e.g., gases, liquefied gases, etc.) can be injected into the headspace, if pCO₂ is elevated, as described with reference to other embodiments herein. Aeration of the bioreactor contents with the headspace gas (not shown) may reduce CO₂ mass transfer limitation for high biomass levels.

According to some embodiments, the cultivation of high levels of bioleaching microbes is helpful for rapid start-up of biomining operations. The sulfide oxidizing bacteria and archaea typically identified as the principal microbes responsible for bioleaching of precious metals can be cultivated in a modified bioreactor, as shown in FIG. 8. This bioreactor is configured in a similar manner as the bioreactor designed for cultivation of the nitrifiers. However, in some embodiments, this bioreactor is supplemented with the addition of inorganic sulfur compounds.

The cultivation of high levels of precious metals precipitating microbes may also be of interest to the biomining industry. As shown in FIG. 9, the bioreactor comprises at least some features of bioreactors used for cultivating nitrifying bacteria and/or bioleaching microbes. Hydrogen gas can be provided to the headspace as an electron donor for these microbes. In some embodiments, a metal solution is provided to the bioreactor to promote the expression of enzymes necessary for bioprecipitation of a specific precious metal.

### Cultivation of Anaerobic Autotrophs

According to some embodiments, for the cultivation of strict anaerobic, autotrophic microbes, gases and solutions that are oxygen free or substantially oxygen free are provided to the corresponding bioreactors. By way of example, Anammox bacteria require strict anaerobic conditions. In some embodiments, a bioreactor, such as, for example, the one schematically illustrated in FIG. 10, can ensure optimal or enhanced growth conditions. In such an embodiment, the inlet gases, e.g., CO₂, N₂, Argon, other gas, combinations thereof, etc., are passed through an O₂ scavenger process prior to injection into the headspace. In addition, in some embodiments, a reducing agent, such as sodium sulfide, sodium cysteine and/or the like, is used to ensure a low oxidation-reduction potential (ORP). An ORP probe may be used to control the bioreactor ORP by the addition of the reducing agent, as needed.

One embodiment of the cultivation of methanogens in a bioreactor with the modifications necessary for headspace pCO₂ control is schematically illustrated in FIG. 11. In such systems, anaerobic gases are injected into the headspace to maintain optimal or enhanced growth conditions for methanogens. As discussed herein, in some embodiments, gases are filter sterilized and oxygen is removed to eliminate contamination and ensure anaerobic operation, respectively. In such a system, the growth substrate can be hydrogen gas, which is used as the electron donor by the methanogens. A hydrogen probe in the headspace can be used to ensure that the pH₂ is maintained at a level that prevents or reduces the likelihood of substrate limitation. Nitrogen gas, which in some embodiments is filter sterilized and oxygen removed, can be used to dilute the headspace gas concentration of CO₂, if necessary. In some embodiments, the methanogens will utilize the H₂ and CO₂ to generate methane. In some configurations, nitrogen gas may not be needed, except for the initial flushing of the headspace. In these systems, the methane content in the headspace can increase over time and excess headspace pressure can be relieved by the transfer of exhaust gas. However, the injection of nitrogen gas into the headspace may reduce the risk of explosion by reducing the hydrogen and methane concentration in the headspace. In some embodiments, at maximum cell density, the methanogen biomass can be removed from the bioreactor by using anaerobic methods. Accordingly, the methanogen biomass can be post-processed for the manufacture of a probiotic or concentrated and stored anaerobically and refrigerated for use as an additive in environmental systems, such as anaerobic digesters or animal waste lagoons.

One embodiment of the cultivation of acetogens in a bioreactor with the modifications necessary for headspace pCO₂ control is schematically illustrated in FIG. 12. This is a similar system to FIG. 8, except the acetate concentration in the bioreactor liquid needs to be controlled by replacement of the bioreactor liquid with nutrient solution when the acetate concentration becomes inhibitory.

One embodiment of the cultivation of aceticlastic methanogens in a bioreactor with the modifications necessary for headspace pCO₂ control is schematically illustrated in FIG. 13. This is a similar system to the one illustrated in FIG. 8, except acetate is added to the bioreactor to prevent substrate limitation of the aceticlastic methanogen. Some aceticlastic methanogens have the capability to reduce CO₂, so H₂ can be injected into the headspace to prevent substrate limitation and promote growth.

According to some embodiments, in order to cultivate syntrophic bacteria and methanogens, a modified UASB reactor, such as the one schematically illustrated in FIG. 14, is utilized. In a typical UASB, the biomass can form granules that are suspended in the tank due to the low velocity fluid directed upwardly from the bottom of the reactor. In a modified UASB reactor, the feed media can comprise nutrients, pH buffer, reducing agent, propionic acid that has been prepared anaerobically and sterilized and/or other components, as desired or required by a particular application or use. The headspace gas content can be controlled for pCO₂ in accordance with any of the embodiments described herein. Nitrogen and/or other gases may be used to dilute the headspace gas content and reduce risk of explosion due to methane content. In some embodiments, when operating properly, the hydrogen content is negligible or substantially negligible. In such a modified UASB reactor, a port can be additionally provided to allow for granule removal. In some embodiments, granules are removed anaerobically and optionally post-processed to generate a probiotic for animals or humans or an additive to environmental systems, such as anaerobic digesters or animal waste lagoons.

Strict anaerobic, autotrophic bacteria that are capable of fermenting Syngas may grow faster in a bioreactor configured to provide optimal or enhanced sCO₂ as shown in FIG. 15.

In some embodiments, for the cultivation of autotrophic, dehalogenating bacteria, a modified bioreactor, such as the one illustrated in FIG. 16, can be used to provide optimal or enhanced sCO₂ concentration. In such a bioreactor, the halogenated organic(s) of interest are transferred to the bioreactor. Dehalogenating bacteria can use H₂ as the electron donor; however, soluble electron donors, such as formate, lactate, benzoate, pyruvate and/or the like can also be used, either in addition to or lieu of H₂. Inorganic electron acceptors, such as sulfate, may be a cost-effective strategy to increase biomass. In some embodiments having high levels of biomass, halogenated organics can be provided as the electron acceptor instead of the inorganic electron acceptor to help ensure the expression of enzymes for dehalogenation.

The cultivation of SCFA methylating microbes can be optimized or enhanced in a modified bioreactor, such as, for example, the one illustrated schematically in FIG. 17. In some embodiments, the substrate SCFAₙ with n carbon atoms is provided to the bioreactor. Further, H₂ and CO₂ can be injected into the headspace to maintain optimal or enhanced growth conditions. Effluent containing high levels of SCFA₍ₙ₊₁₎ may be available for recovery. With proper handling of the anaerobic biomass, the microbes can be processed for use as a probiotic for human and animal health or environmental systems, such as, for example, anaerobic digesters or waste lagoons.

FIG. 18 schematically illustrates one embodiment of a bioreactor that can be used for the optimal or enhanced cultivation of alkane methylating microbes. In such an arrangements, the substrate Alkaneₙ with n carbon atoms is provided to the bioreactor in the gaseous form (n=2-4) or soluble form (n>4). H₂ and CO₂ are injected into the headspace to maintain optimal growth conditions. Exhaust gas or effluent containing high levels of Alkane₍ₙ₊₁₎ may be available for recovery. The alkane methylating microbe may be recovered by using proper anaerobic handling. In some embodiments, this microbe is used for bioaugmentation of anaerobic digesters, landfills, coalbeds, and other natural or engineered systems where methane is generated and/or the like.

According to some embodiments, the cultivation of alcohol methylating microbes can be optimized or enhanced using a modified bioreactor, such as, for example, the one schematically illustrated in FIG. 19. In some arrangements, the substrate Alcoholₙ with n carbon atoms is provided to the bioreactor in the soluble form. H₂ and CO₂ can be injected into the headspace to maintain optimal growth conditions. Effluent containing high levels of Alcohol₍ₙ₊₁₎ may be available for recovery. The alcohol methylating microbe may be recovered by using proper anaerobic handling. This microbe may be used for post-proces sing of ethanol generating plants.

According to some embodiments, the cultivation of methanogens in a separate bioreactor can be used to bioaugment an anaerobic digester and enrich the CH₄ content of the anaerobic digester biogas. One such embodiment is schematically illustrated in FIG. 20. In some embodiments, H₂ and anaerobic digester biogas containing CO₂ can be injected into the headspace to maintain optimal or enhanced growth conditions within the bioreactor. Acetate, nutrients, reducing agent, and pH buffer can be added to the methanogen bioreactor to maintain optimal or enhanced growth conditions for aceticlastic methanogens. Filter sterilized primary clarifier effluent without dissolved oxygen can be used as the replacement liquid in the bioreactor. Bioreactor effluent containing high levels of methanogens may be available for bioaugmentation of the anaerobic digester. CH₄-enriched biogas can be transferred to the headspace of the anaerobic digester to advantageously reduce the pCO₂ of the anaerobic digester and subsequently improve the growth rate of autotrophs in the anaerobic digester.

For higher organic loading rates of a conventional anaerobic digester, hydrogen (and acetate and CO₂) generation rates can increase and more hydrogen can be available to both CO₂-reducing methanogens and acetogens. Acetogens typically have a very flexible metabolism that allows for fermentation of carbohydrates or CO₂ reduction to form acetate as the endproduct. In some embodiments, within the normal range of organic loading rates for conventional anaerobic digesters, the rate of acetate production by fermentation does not exceed the rate of biomethane production by aceticlastic methanogens. Aceticlastic methanogens generate about ⅔ of the methane for operation within the normal organic loading rates for anaerobic digesters fed sewage sludges. In some embodiments, CO₂-reducing acetogens are not considered to be a significant pathway for hydrogen when anaerobic digesters are operated within normal organic loading rates. In some arrangements, CO₂-reducing acetogens may compete for hydrogen when low pH conditions (i.e., sour digester) are present, resulting in more acetate being generated and lower pH. In some embodiments, if higher organic loading rates are desired, higher levels of methanogens may be needed to maintain stable operation. In particular, the expected higher levels of acetate due to higher organic loading rates may require much higher level of aceticlastic methanogens, because their specific growth rate is generally slower compared to the fermenting bacteria and CO₂-reducing methanogens.

In some embodiments, bioaugmentation of the anaerobic digester with aceticlastic methanogens cultivated in the bioreactor improves the overall biomethane generation rate in the anaerobic digester. In some embodiments, bioaugmentation artificially increases the abundance of aceticlastic methanogens, which would compensate for their slower specific growth rate. Thus, in some arrangements, by maintaining high biomethane generate rates via, for example, the aceticlastic methanogens, acetate levels would not buildup and cause a drop in pH. However, this approach would typically require the purchase of acetate. In addition, the reduction in CO₂ from the biogas may be limited to biomass generation (i.e., anabolism). In this case, high levels of aceticlastic methanogen biomass may be required to have a substantial impact on the pCO₂ of the anaerobic digester for improving the specific growth rates of the autotrophs, which, under certain circumstances, could be cost prohibitive. However, some aceticlastic methanogens, such as Methanosarcina barkeri, can also reduce CO₂ with available hydrogen. The cultivation of aceticlastic methanogens with this metabolic capability would be one preferred embodiment of this approach, since CO₂ from the biogas could be utilized for both anabolism and catabolism. Under such embodiments, when transferred to the anaerobic digester, the methanogens would be available for either CO₂ reduction or acetate utilization depending on which substrate is available.

Such a bioaugmentation strategy could also allow for much higher organic loading rates, which may be possible when sludge hydrolyzing processes are used to pretreat feed sewage sludges or other organic solids. Currently, in some circumstances, organic loading rates of pre-hydrolyzed organics are limited due to the inability of slow-growing methanogens to rapidly utilize available acetate or H₂ when exposed to elevated pCO₂ in the anaerobic digester headspace. Efficient hydrolysis of sewage sludges also has the advantage of reducing the pathogen content of the biosolids and may allow for reduced solids residence time in the anaerobic digester. Thus, in some circumstances, the capital costs of the anaerobic digester system can be significantly reduced due to operating at the lower solids residence time. On the other hand, capital costs of the hydrolysis process and bioreactor can, in certain circumstances, increase the overall cost. In some circumstances, the operational costs of the bioreactor increase the costs of generating biomethane due to the extra hydrogen required. However, the biomethane quality may be improved and the costs associated with CO₂ removal or natural gas addition can be reduced or eliminated. In some circumstances, the bioaugmentation of an anaerobic digester having relatively high levels of methanogens can increase the steady-state concentration of methanogens, which can provide a competitive advantage for methanogens over sulfate reducing bacteria (SRB) for available hydrogen. In conventional anaerobic digesters, for example, the SRB outcompete the methanogens for available hydrogen and convert any available sulfate or sulfur to hydrogen sulfide. This can decrease the quality of the biogas and add to the cost for hydrogen sulfide removal prior to use. With the use of the methanogen bioreactor, the methanogens can, in certain embodiments, outcompete the SRB for available hydrogen based on the relatively large difference in their biomass concentration. Under such conditions, the level of hydrogen sulfide in the biomethane will be much lower or eliminated, if the SRB are washed out of the anaerobic digester.

### Evaluation and Improvement of Growth Conditions for Autotrophs in Wastewater and Sludge Treatment Systems

In order to ensure performance improvement by bioaugmentation, the evaluation of the growth conditions for autotrophs in wastewater and sludge treatment systems would be helpful. Ideally, the growth conditions of the autotrophic microbe with respect to pH and sCO₂ would be in close agreement between the bioreactor used for cultivation of the bioaugmentation product and the targeted wastewater or sludge treatment system. In some cases, the direct measurement of the sCO₂ concentration may be cost prohibitive. However, the total alkalinity of the wastewater or sludge treatment system can be measured with inexpensive methods (i.e., chemical test strips or acid titration), which can be used with the pH to calculate the sCO₂ concentration.

Ideally, historical data could be used to evaluate growth conditions in the targeted wastewater or waste treatment system prior to bioaugmentation. The inefficient growth conditions could then be improved using supplemental CO₂ in the aeration system for increasing the sCO₂ concentration or increasing the aeration rate for reducing the sCO₂ concentration. Predictable changes in the growth conditions that shift the dominance of competing autotrophs resulting in short-term poor performance may also be dampened by bioaugmentation that rapidly increases the biomass of the dominant autotroph that is at low abundance. Another option for increasing the sCO₂ concentration would be the retrofit of lift stations of the collection system. A simple air-tight enclosure may be used to allow for the control of the pCO₂ in the lift station by the use of an air pump, pH probe, and periodic measurement of the total alkalinity.

### Non-Selective Growth Conditions for Autotrophs in Wastewater and Sludge Treatment Systems

In some cases, the growth of some autotrophic microbes is not of interest. For example, secondary treatment systems may be interested in reducing nitrification in order to improve sludge settling in the secondary clarifier and reduce nitrite levels for reduced chlorine demand. Operation at an elevated sCO₂ concentration and associated low pH would reduce the specific growth rate of both pairs of nitrifying bacteria. This approach may be of interest for the latter half of the aeration basin, where the bulk of the BOD removal has been observed. Another approach would be alternating operation at two extreme sCO₂ concentrations in the aeration basin to reduce the growth of both pairs of nitrifying bacteria. Both approaches would reduce the overall rate of nitrification and subject them to eventual washout from the activated sludge system. In some cases, intense aeration may be necessary to reduce the sCO₂ concentration and increase the pH prior to discharge to receiving water. In another example, the lift stations could also be operated at high sCO₂ concentration in order to reduce the rate of sulfate reduction for odor control and crown corrosion.

### Biological Nitrogen Removal System

With the measurement of both total alkalinity and pH, biological wastewater treatment systems can be configured and operated to enhance the growth of select autotrophic microbes by control of the sCO₂ concentration. In the simplest design (FIG. 23), the two aeration banks are operated at two extreme sCO₂ concentrations for the purpose of controlling nitrification. The two sCO₂ concentration set points are determined by the total alkalinity and the growth sensitivities of the two nitrifying bacteria pair (FIGS. 21 and 22). Operation at the elevated sCO₂ concentration of about 35 mg/L in the first aeration basin will limit the extent of nitrification when the entire system is operated at a low SRT. BOD removal by the heterotrophic biomass will not be impacted by the elevated sCO₂ concentration. The first aeration basin effluent with elevated sCO₂ concentration would then be split with one half aerobically treated for nitrite formation at low sCO₂ concentration (and associated high pH) in the AOB reactor. Intense aeration in the AOB reactor will strip the CO2 and increase the pH necessary for rapid ammonium oxidation by the ammonium oxidizing bacteria (AOB) to convert the ammonium to nitrite. The nitrite-rich wastewater from the AOB reactor can be combined with the ammonium-rich wastewater from the first aeration tank and treated in the Anammox reactor, which would not be aerated (i.e., anaerobic and optimal sCO₂). In the Anammox reactor, a blend of equal parts ammonium and nitrite is converted under anoxic conditions to nitrogen gas. According to some embodiments, if anaerobic conditions are difficult to maintain in the Anammox reactor due to the destruction of strict anaerobic bacteria, a small flow rate (e.g., approximately 1%, 0.5-2%, 2-5%, 5-10%, etc.) of primary solids or raw wastewater (e.g., as shown in FIGS. 23-25 as the dashed line) may be periodically or continuously provided to the Anammox reactor to ensure strict anaerobic conditions.

In some wastewater treatment systems, maintaining an enhanced concentration of soluble CO₂ in the Anammox reactor may be difficult due to CO₂ generation from the anaerobic biodegradation of residual BOD or decay of biomass. Although nitrogen gas is generated by the Anammox bacteria, industrial nitrogen gas could be also used to prevent the increase of pCO₂ in the headspace.

According to some embodiments, headspace gas within a bioreactor is used for gas mixing, either instead of or in lieu of mechanically mixing the biomass and wastewater in the Anammox reactor. Excess gas in the headspace can be removed by a pressure relief valve and/or any device or method. In some embodiments, since heterotrophic bacteria, AOB, and Anammox bacteria grow relatively rapidly, the solids residence time (SRT) of the system does not need to be maintained at relatively high values typical of systems designed for nitrogen removal. To ensure proper settling of the activated sludge in the secondary clarifier, the SRT can be maintained at a value of about 5 days (e.g., 3, 4, 5, 6, 7, 8 days, less than 3 days, more than 8 days, time periods between the foregoing values, etc.), which is comparable to a typical activated sludge system designed for BOD removal. Operation at this lower SRT can also help ensure that nitrite oxidizing bacteria are at very low concentrations due to the washout pressure.

### Biological Nutrient Removal System

In some embodiments of wastewater treatment systems, phosphorus removal may also be desired. One embodiment of a modified Biological Nitrogen System is shown in FIG. 24. In this configuration, an anaerobic basin with pCO₂ control is used to treat wastewater and return activated sludge (RAS). In some embodiments, under anaerobic conditions, the PAO can release phosphorus and take up volatile fatty acid and store it as an organic storage polymer, such as polyhydroxybutryate (PHB). The remainder of the treatment train can be identical to that illustrated in FIG. 23 and discussed herein. In the first aeration basin, the BOD can be oxidized by heterotrophic biomass, and the PAO can take up the phosphorus by aerobically metabolizing the PHB or other organic storage polymer. In some embodiments, nitrification is limited by operation at an elevated sCO₂ concentration. According to some embodiments, similar to the Biological Nitrogen Removal system (FIG. 23), the SRT of this system can be maintained at a value of about 5 days to reduce the level of the nitrifying bacteria besides the AOB. If anaerobic conditions are difficult to maintain in the Anammox reactor, then the strategy described for the Biological Nitrogen Removal System (FIG. 23) may be employed.

### Biomethane Generation and Biological Nutrient Removal System

Another embodiment of a treatment system utilizing one or more methods and/or bioreactor concepts discussed herein is schematically illustrated in FIG. 25. In some embodiments, a side stream biomethane reactor (see FIG. 20) can be used to bioaugment the anaerobic basin with aceticlastic methanogens. Hydrogen gas can be used to generate additional methane from the carbon dioxide produced by fermentation and aceticlastic methanogenesis in the anaerobic basin. The generation of biomethane and recirculation of the biomethane into the headspace of the anaerobic basin can help ensure that enhanced (e.g., improved, optimal, etc.) sCO₂ conditions are provided for methanogenesis. According to some configurations, the side stream biomethane reactor can be advantageously operated within a desired temperature or range. In some embodiments, the anaerobic basin can be operated under ambient temperatures, but the enhanced sCO₂ concentration can counter the inhibition of the lower operating temperature. The rest of the treatment system is similar to FIG. 24. If anaerobic conditions are difficult to maintain in the Anammox reactor, then the strategy described in the Biological Nitrogen Removal System (FIG. 23) may be employed.

### Design and Operation of Wastewater Treatment Systems for Maintaining Enhanced Growth Conditions of Autotrophic Microbes

The enhancement (e.g., optimization) of the growth conditions for the autotrophic microbes in wastewater treatment systems can be accomplished by combining the knowledge of the sensitivity of the specific growth rate of different types of autotrophic microbes to pH and sCO₂, the various methods for sCO₂ control, measurements of key wastewater characteristics, such as flow rate, pH, total alkalinity, ammonium, temperature, and sCO₂. In some embodiments, these measurements can be used with a SCADA system to provide real-time control of the growth conditions of the autotrophic microbes in specific basins of the treatment train by adjustment of sCO₂. Furthermore, the design of wastewater treatment systems to optimize the growth of autotrophic microbes is also possible through the use of advanced mathematical modeling software that incorporates real-time sCO₂ control within the treatment train. Prior to retrofits of wastewater treatment systems for enhancing the growth conditions of autotrophic microbes, historical data of influent wastewater characteristics can be used to provide insight into temporal changes that inhibit growth. In this way, a retrofit based on the inventions disclosed herein may be designed to counter these influent changes and provide enhanced (e.g., optimal, improved, etc.) performance of the existing infrastructure.

Although these inventions have been disclosed in the context of certain preferred embodiments and examples, it will be understood by those skilled in the art that the present inventions extend beyond the specifically disclosed embodiments to other alternative embodiments and/or uses of the inventions and obvious modifications and equivalents thereof. In addition, while the number of variations of the inventions have been shown and described in detail, other modifications, which are within the scope of these inventions, will be readily apparent to those of skill in the art based upon this disclosure. It is also contemplated that various combinations or subcombinations of the specific features and aspects of the embodiments may be made and still fall within the scope of the inventions. Accordingly, it should be understood that various features and aspects of the disclosed embodiments can be combined with, or substituted for, one another in order to perform varying modes of the disclosed inventions. Thus, it is intended that the scope of the present inventions herein disclosed should not be limited by the particular disclosed embodiments described above, but should be determined only by a fair reading of the claims.

What is claimed is:

1. A method for controlling the growth of autotrophic cells in a bioreactor, comprising:
determining a concentration of soluble carbon dioxide within a liquid mixture of the bioreactor, wherein the bioreactor comprises a volume of the liquid mixture below a headspace of the bioreactor;
wherein said liquid mixture comprises autotrophic cells and substrate, said substrate being configured to promote the growth of the autotrophic cells when the bioreactor is in operation;
calculating a target range of a concentration of soluble carbon dioxide within the liquid mixture based on, at least in part, on empirical or experimental data, wherein the target range provides for controlled growth of the autotrophic cells when the bioreactor is in use;
comparing the target range of the concentration of soluble carbon dioxide within the liquid mixture to the concentration of soluble carbon dioxide in the liquid mixture; and
adjusting the concentration of soluble carbon dioxide within the liquid mixture by at least one of: (i) modifying a partial pressure of carbon dioxide gas within the headspace, and (ii) modifying the concentration of soluble carbon dioxide within the liquid mixture by delivering a volume of a supplement stream to the liquid mixture;
wherein a concentration of soluble carbon dioxide in the supplement stream is different than the concentration of soluble carbon dioxide of the liquid mixture.

2. The method of claim 1, wherein the headspace is not in fluid communication with an ambient environment, such that an interior of the bioreactor comprises a closed system.

3. The method of claim 1, wherein the headspace is in fluid communication with an ambient environment, such that the bioreactor comprises an open system.

4. A method of claim 1, further comprising measuring a temperature of the liquid mixture, wherein the measured temperature of the liquid mixture is used, at least in part, to calculate the target range of a concentration of soluble carbon dioxide.

5. The method of claim 4, further comprising modifying a temperature of the liquid mixture to achieve a target temperature for the liquid mixture, wherein the target temperature assists in enhancing the growth of the autotrophic cells.

6. A method of claim 1, further comprising measuring a pH of the liquid mixture, wherein the measured pH is used, at least in part, to calculate the target range of a concentration of soluble carbon dioxide.

7. The method of claim 6, further comprising modifying a pH of the liquid mixture to achieve a target pH for the liquid mixture, wherein the target pH assists in enhancing the growth of the autotrophic cells.

8. A method of claim 1, wherein modifying the partial pressure of carbon dioxide gas within the headspace comprises introducing a volume of gas within the headspace of the bioreactor, the volume of gas comprising a concentration of carbon dioxide that is different than a concentration of carbon dioxide gas within said headspace.

9. The method of claim 8, wherein the gas introduced within the headspace comprises an inert gas having little or no carbon dioxide.

10. The method of claim 9, wherein the inert gas comprises nitrogen (N₂).

11. A method of claim 1, wherein determining the concentration of soluble carbon dioxide within the liquid mixture comprises using a probe.

12. The method of claim 11, wherein the probe is inserted or positioned within the liquid mixture to directly determine the concentration of soluble carbon dioxide.

13. A method of claim 1, wherein the bioreactor is incorporated into an engineered biological system.

14. The method of claim 13, wherein the engineered biological system comprises a wastewater treatment system.

15. The method of claim 14, wherein the bioreactor is in fluid communication with a treatment chamber of the wastewater treatment system so that autotrophic cells grown within the bioreactor can be selectively delivered into the treatment chamber.

16. A method of claim 1, wherein controlled growth of autotrophic cells comprises at least one of enhancing or inhibiting growth of said cells.

17. A bioreactor for controlling the growth of autotrophic cells, comprising:
at least one chamber for retaining a liquid mixture
an inlet and an outlet in fluid communication with the at least one chamber, wherein the inlet in configured to permit a liquid mixture to enter the bioreactor, and wherein the outlet is configured to permit a liquid mixture to exit the bioreactor; and
a headspace located above the chamber and the liquid mixture;
at least one probe or sensor configured to determine a concentration of soluble carbon dioxide within a liquid mixture of the bioreactor;
a gas regulation system configured to permit a gas to be selectively moved within the headspace of the bioreactor, wherein the gas regulation system is configured to: (i) alter a concentration of carbon dioxide of the gas moved within the headspace and/or (ii) alter a flowrate of the gas moved within the headspace;
a control system for regulating a concentration of soluble carbon dioxide within the liquid mixture, wherein the control system is configured to determine a target range of a concentration of soluble carbon dioxide within the liquid mixture based on, at least in part, on empirical or experimental data, wherein the target range provides for controlled growth of the autotrophic cells when the bioreactor is in use;
wherein the control system is configured to compare the target range of the concentration of soluble carbon dioxide within the liquid mixture to the concentration of soluble carbon dioxide in the liquid mixture; and
wherein the control system is configured to adjust the concentration of soluble carbon dioxide within the liquid mixture by at least one of: (i) modifying a partial pressure of carbon dioxide gas within the headspace, and (ii) modifying the concentration of soluble carbon dioxide within the liquid mixture by delivering a volume of a supplement stream to the liquid mixture;

18. A bioreactor of claim 17, wherein the bioreactor further comprises at least one additional probe or sensor.

19. A bioreactor of claim 17, wherein the bioreactor is incorporated into a wastewater treatment system.

20. The bioreactor of claim 17, wherein the bioreactor comprises an activated sludge treatment tank and/or an anaerobic digester included in a treatment scheme.
